(19) United States
(12) Patent Application Publication
(10) Pub. No.: US 2009/0209547 A1
(43) Pub. Date: Aug. 20, 2009

(54) C-8 HALOGENATED MACROLIDES

(76) Inventors: IN JONG KIM, LEXINGTON, MA (US); GUOQIANG WANG, BELMONT, MA (US); HEEJIN KIM, ALLSTON, MA (US); YAT SUN OR, WATERTOWN, MA (US)

Correspondence Address: ELMORE PATENT LAW GROUP, PC, 515 Groton Road, Unit 1R, Westford, MA, 01886, US

(21) Appl. No.: 12/370,805

(22) Filed: Feb. 13, 2009

Related U.S. Application Data
(60) Provisional application No. 61/029,313, filed on Feb. 15, 2008.

(51) Int. Cl. A61K 31/4985; A61K 31/4375; A61K 31/4439; A61K 31/424; C07D 471/04; C07D 498/04

(52) U.S. Cl. 514/250; 514/300; 514/338; 514/375; 544/350; 546/122; 546/271.7; 548/218

(57) ABSTRACT

The present invention discloses compounds of formula (I) or pharmaceutically acceptable salts, esters, or prodrugs thereof:

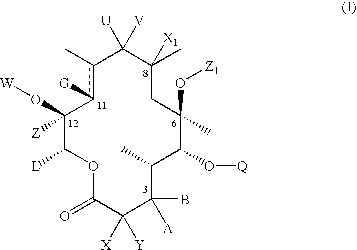

which exhibit antibacterial properties. The present invention further relates to pharmaceutical compositions comprising the aforementioned compounds for administration to a subject in need of antibiotic treatment. The invention also relates to methods of treating a bacterial infection in a subject by administering a pharmaceutical composition comprising the compounds of the present invention. The invention further includes process by which to make the compounds of the present invention.

### RELATED APPLICATIONS

This application claims the benefit of U.S. provisional application No. 61/029,313 filed on Feb. 15, 2008. The contents of the above identified application are incorporated herein by reference.

### TECHNICAL FIELD

The present invention relates to novel semisynthetic macrolides having antibacterial activity that are useful in the treatment and prevention of bacterial infections. More particularly, the invention relates to C8 halogenated compounds, compositions containing such compounds and methods for using the same, as well as processes for making such compounds.

### BACKGROUND OF THE INVENTION

The spectrum of activity of macrolides, including erythromycin, covers most relevant bacterial species responsible for upper and lower respiratory tract infections. 14-membered ring macrolides are well known for their overall efficacy, safety and lack of serious side effects. Erythromycin however is quickly degraded into inactive products in the acidic medium of the stomach resulting in low bioavailability and gastrointestinal side effects. Improvement of erythromycin pharmacokinetics has been achieved through the synthesis of more acid-stable derivatives, for example, roxithromycin, clarithromycin, and the 15-membered ring macrolide azithromycin. However, all of these drugs, including 16-membered ring macrolides, present several drawbacks. They are inactive against MLS_{B}-resistant streptococci (MLS_{B}=Macrolides-Lincosamides-type B Streptogramines) and with the exception of azithromycin, weakly active against Haemophilus influenzae. Furthermore, the resistance of Streptococcus pneumoniae to erythromycin has increased significantly in recent years (5% to above 40%). There is a high percentage of cross-resistance to penicillin among these isolates, with a worldwide epidemic spread of 10-40% in some areas.

In recent years, there has been an increased demand for new anti-MRSA (methicillin resistant staphylococcus aureus) agents, particularly with the rising incidence of community acquired MRSA. Since the currently available macrolides and ketolides are not active against most MRSA and constitutive resistant S. aureus, there is a clear need for new macrolides that overcome the problem of pneumococcal and staphylococcus resistance, have good pharmacokinetic properties and acid stability while continuing to be active against H. influenzae. These new macrolides will be ideal candidates for drug development in the first line therapy of upper respiratory tract infections (“URTI”) and lower respiratory tract infections (“LRTI”).

Kashimura et al. have disclosed 6-O-methylerythromycin derivatives having a tricyclic basic nuclear structure in European Application 559896, published Nov. 11, 1991. Also, Asaka et al. have disclosed 5-O-desoaminylerythronolide derivatives containing a tricyclic carbamate structure in PCT Application WO 93/21200, published Apr. 22, 1992.

Erythromycin derivatives containing a variety of substituents at the 6-O position have been disclosed in U.S. Pat. Nos. 5,866,549 and 6,075,011 as well as PCT Application WO 00/78773. Furthermore, Ma et al. have described erythromycin derivatives with aryl groups tethered to the C-6 position in J. Med. Chem., 44, pp 4137-4156 (2001). PCT application WO 97/10251, published Mar. 20, 1997, discloses intermediates useful for preparation of 6-O-methyl 3-descladinose erythromycin derivatives. U.S. Pat. Nos. 5,866,549 and 6,075,011, and PCT application WO 00/78773, published Dec. 28, 2000, disclose certain 6-O-substituted erythromycin derivatives.

### SUMMARY OF THE INVENTION

The present invention provides a novel class of C8 halogenated compounds that possess antibacterial activity.

The compounds of the present invention are represented by formula (I) as illustrated below:

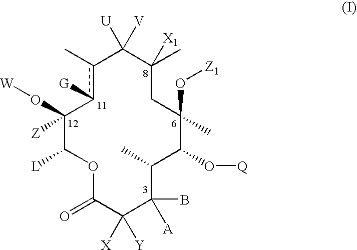

or the racemates, enantiomers, diastereomers, geometric isomers, tautomers, solvates, pharmaceutically acceptable salts, esters and prodrugs thereof, wherein one of U and V is hydrogen or hydroxy and the other is selected from:
(a) hydrogen;
(b) —OR₁; where R₁ is independently selected from the group consisting of:
   (i) hydrogen;
   (ii) aryl; substituted aryl; heteroaryl; substituted heteroaryl;
   (iii) —R₂, where R₂ is substituted or unsubstituted —C₁-C₈ alkyl, —C₂-C₈ alkenyl, or —C₂-C₈ alkynyl each containing 0, 1, 2, or 3 heteroatoms selected from O, S or N; and
   (iv) —R₃, where R₃ is substituted and unsubstituted —C₃-C₁₂ cycloalkyl each containing 0, 1, 2, or 3 heteroatoms selected from O, S or N;
(c) —R₂;
(d) —OC(O)NHR₁;
(e) —OC(O)OR₁;
(f) —NR₄R₅; where R₄ and R₅ are each independently selected from R₁; alternatively, R₄ and R₅ taken together with the nitrogen atom to which they are connected form a 3- to 10-membered ring which may optionally contain one or more heterofunctions selected from the group consisting of: —O—, —NH—, —N(C₁-C₈-alkyl)-, —N(R₆)—, —S(O)ₙ—, wherein n=0, 1 or 2, and R₆ is selected from aryl; substituted aryl; heteroaryl; and substituted heteroaryl;
(g) —NHC(O)R₁;
(h) —NHS(O)₂R₁;
(i) —NHC(O)OR₁; and
(j) —NHC(O)NHR₁;
   alternatively, U and V taken together with the carbon atom to which they are attached are selected from:

(a) C(O);

(b) C═N-J-R₁, where J is absent, O, C(O), S(O)₂, NH, NHC(O), NHC(O)NH or NHS(O)₂;

(c) C═CH-J-R₇; wherein R₇ is independently selected from halogen and R₁;

(d) substituted or unsubstituted, saturated or unsaturated 5- to 10-membered heterocyclic;

X₁ is halogen;
Z₁ is independently selected from:
(a) aryl; substituted aryl; heteroaryl; substituted heteroaryl;
(b) substituted or unsubstituted —C₁-C₈ alkyl, —C₂-C₈ alkenyl, or —C₂-C₈ alkynyl each containing 0, 1, 2, or 3 heteroatoms selected from O, S or N; and
(c) substituted or unsubstituted —C₃-C₁₂ cycloalkyl, or —C₃-C₁₂ cycloalkenyl each containing 0, 1, 2, or 3 heteroatoms selected from O, S or N;
   or Z₁ and either A or B can be taken together to form cyclic structure

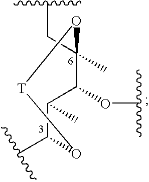

wherein T is
(a) —R₈—, where R₈ is substituted or unsubstituted —C₁-C₈ alkylene-, —C₂-C₈ alkenylene- or —C₂-C₈ alkynylene-, containing 0, 1, 2, or 3 heteroatoms selected from O, S or N;
(b) —R₈—C(O)—R₉—, where R₉ is independently selected from R₈;
(c) —R₈—(C═N-E-R₁)—R₉—, where E is absent, O, NH, NHC(O), NHC(O)NH or NHS(O)₂;
(d) —R₈—[C(OR₁₀)(OR₁₁)]—R₉—, where R₁₀ and R₁₁ are independently selected from the group consisting of C₁-C₁₂ alkyl, aryl or substituted aryl; or R₁₀ and R₁₁ taken together is —(CRₐR_{b})ᵣ—, where r is 2 or 3; Rₐ and R_{b} are independently selected from hydrogen, aryl, and R₂;
(e) —R₈—[C(SR₁₀)(SR₁₁)]—R₉—; or
(f) —R₈—(C═CH—R₁)—R₉—;
   G is selected from the group consisting of:

a) hydrogen;

b) hydroxy;

c) —O—R₂; and

W is selected from:

(a) hydrogen;

(b) —R₂;

(c) —C(O)R₁;

(d) —C(O)O—R₁; and

(e) —C(O)N(R₄R₅);

Alternatively, G and W taken together with the atoms to which they are attached form a cyclic structure selected from:

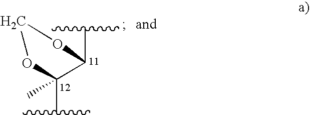

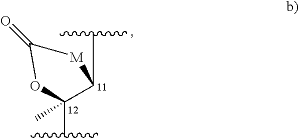

where M is O, —CHR₁ or N-J₁-R₁₂, and where J₁ is absent, O, NH, NHC(O), or N═CH; and R₁₂ is selected from the group consisting of:
i. hydrogen;
ii. R₂; and
iii. R₆;
   one of A and B is R₁₃ and the other is OR₁₃, wherein R₁₃ is independently selected from:

(a) hydrogen;

(b) —R₂;

(c) —C(O)R₁;

(d) —C(O)NHR₁;

(e) —S(O)₂R₁;

(f) -monosaccharide; and

(g) -disaccharide;

alternatively, A and B taken together with the carbon atom to which they are attached form:
(a) C(O);
(b) C═CH-J-R₇, where J is absent, O, C(O), S(O)₂, NH, NHC(O), NHC(O)NH or NHS(O)₂; and wherein R₇ is independently selected from halogen and R₁;
   L is independently selected from R₂;

### Q is:

(a) —R₁;

(b) —C(O)R₁;

(c) —C(O)NHR₁;

(d) —C(O)OR₁;

(e) —S(O)₂R₁;

(f) monosaccharide;

(g) disaccharide; or

(h) trisaccharide;

### Z is:

(a) hydrogen;

(b) —N₃;

(c) —CN;

(d) —NO₂;

(e) —C(O)NH₂;

(f) —C(O)OH;

(g) —CHO;

(h) —R₂;

(i) —C(O)OR₂;

(j) —C(O)R₂; or

(k) —C(O)NR₃R₄;

each of X and Y is independently:

a) hydrogen;

b) hydroxy;

c) halogen; or

d) —R₂; and

[symbol]

=either a carbon-carbon single bond or a carbon-carbon double bond.

In another embodiment of the present invention there are disclosed pharmaceutical compositions comprising a therapeutically effective amount of a compound of the invention in combination with a pharmaceutically acceptable carrier or excipient. In yet another embodiment of the invention are methods of treating antibacterial infections in a subject in need of such treatment with said pharmaceutical compositions. Suitable carriers and formulations of the compounds of the present invention are disclosed.

### DETAILED DESCRIPTION OF THE INVENTION

In a first embodiment of the compounds of the present invention are compounds represented by formula I as illustrated above, or a pharmaceutically acceptable salt, ester or prodrug thereof.

In one embodiment of the compounds of the present invention are compounds represented by formula II as illustrated below, or a pharmaceutically acceptable salt thereof:

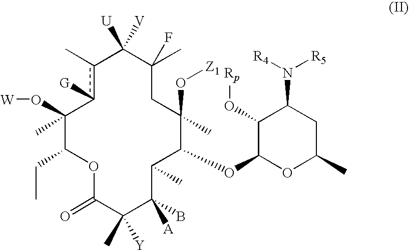

where Rₚ is hydrogen, hydroxy protecting group, ester or hydroxy prodrug; A, B, G, W, U, V Y, R₄, R₅ and Z₁ are as previously defined.

In one embodiment of the compounds of the present invention are compounds represented by formula III as illustrated below, or a pharmaceutically acceptable salt, ester or prodrug thereof:

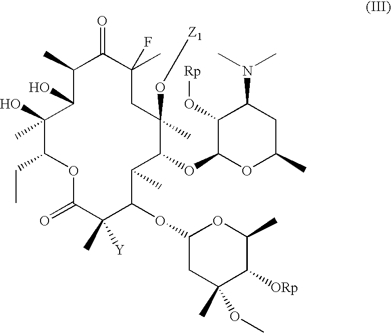

where Z₁, Y and Rₚ are as previously defined.

In one embodiment of the compounds of the present invention are compounds represented by formula IV as illustrated below, or a pharmaceutically acceptable salt, ester or prodrug thereof:

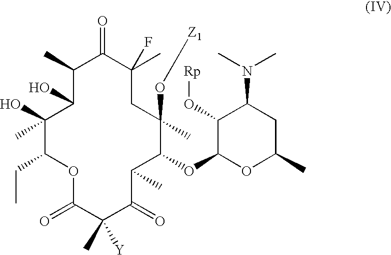

where Z₁, Y and Rₚ are as previously defined.

In one embodiment of the compounds of the present invention are compounds represented by formula V as illustrated below, or a pharmaceutically acceptable salt, ester or prodrug thereof:

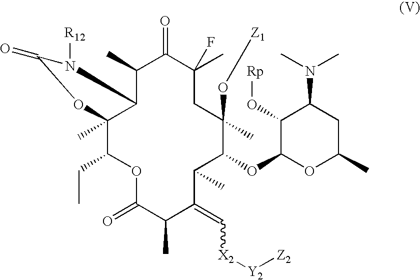

where X₂ is C(O) or CH₂; Y₂ is absent, O, S, NH, C(O), C(O)O, C(O)NH, S(O), S(O)₂, C(S), C(S)NH, OC(O)O, OC(O)NH, OC(O), C(O)O, NHC(O)O, NHC(O), NHC(O)NH, NHS(O)₂, S(O)₂NH, NHS(O)₂)NH, or C═N-E-R₃; Z₂ is hydrogen, R₂ or R₆; and R₁₂, Z₁, Y and Rₚ are as previously defined.

In one embodiment of the compounds of the present invention are compounds represented by formula VI as illustrated below, or a pharmaceutically acceptable salt, ester or prodrug thereof:

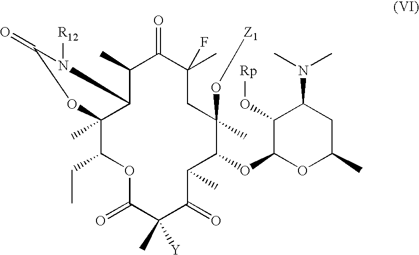

where Z₁, Y, R₁₂ and Rₚ are as previously defined.

In one embodiment of the compounds of the present invention are compounds represented by formula VII as illustrated below, or a pharmaceutically acceptable salt, ester or prodrug thereof:

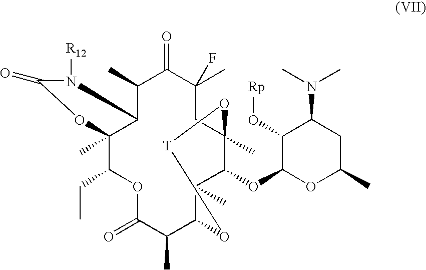

where T, R₁₂ and Rₚ are as previously defined.

Representative compounds according to the invention are those selected from the group consisting of:

compounds (1)-(43) of the formula A:

TABLE 1

| | |
|---|---|
| | (A) |
| 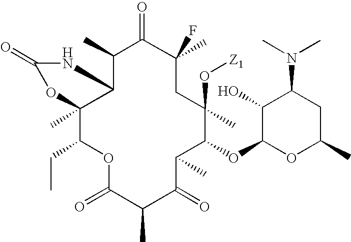 | |

| Compound | Z₁ |
|---|---|
| 1 | CH₃ |
| 2 | 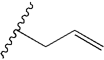 |
| 3 | 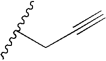 |
| 4 | 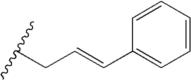 |
| 5 | 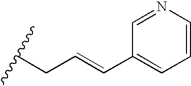 |
| 6 | 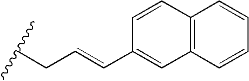 |
| 7 | 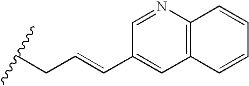 |
| 8 | 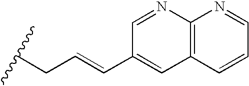 |
| 9 | 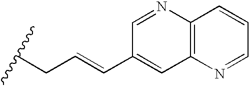 |
| 10 | 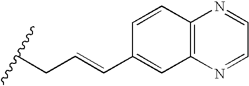 |
| 11 | 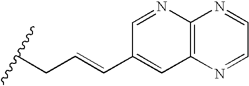 |
| 12 | 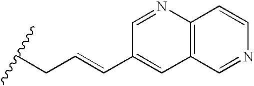 |
| 13 | 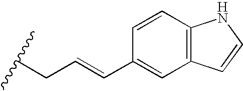 |
| 14 | 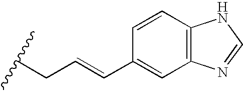 |
| 15 | 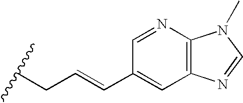 |
| 16 | 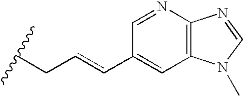 |
| 17 | 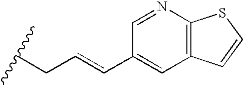 |
| 18 | 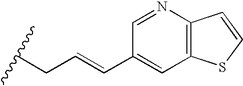 |
| 19 | 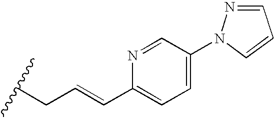 |
| 20 | 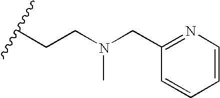 |
| 21 | 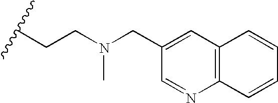 |
| 22 | 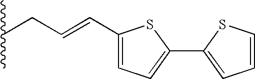 |
| 23 | 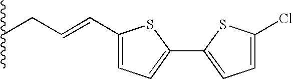 |
| 24 | 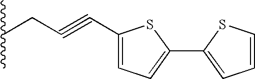 |
| 25 | 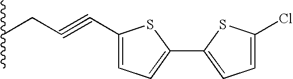 |
| 26 | 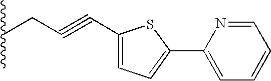 |
| 27 | 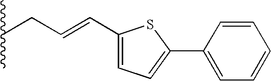 |
| 28 | 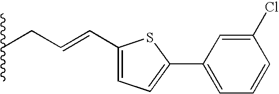 |
| 29 | 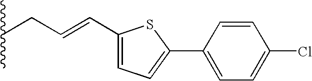 |
| 30 | 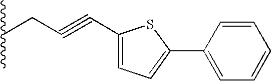 |
| 31 | 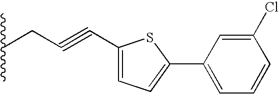 |
| 32 | 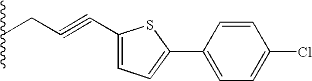 |
| 33 | 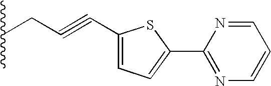 |
| 34 | 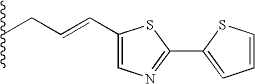 |
| 35 | 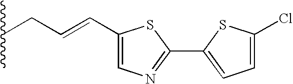 |
| 36 | 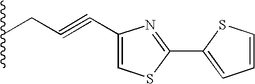 |
| 37 | 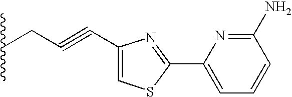 |
| 38 | 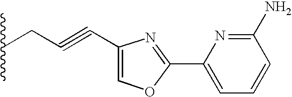 |
| 39 | 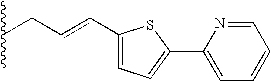 |
| 40 | 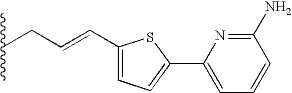 |
| 41 | 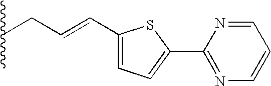 |
| 42 | 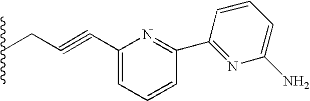 |
| 43 | 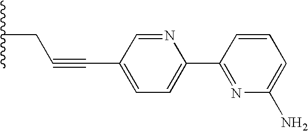 |

Further representative compounds according to the invention are those selected from the group consisting of:

Compounds (44)-(55) of the formula B:

TABLE 2

| | |
|---|---|
| | (B) |
| 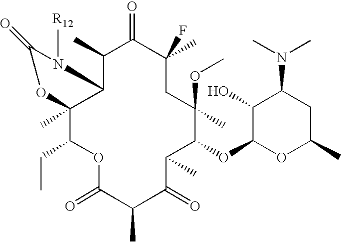 | |

| Com- | |
|---|---|
| pound | R₁₂ |
| 44 | H |
| 45 | 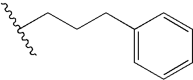 |
| 46 | 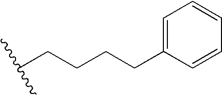 |
| 47 | 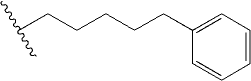 |
| 48 | 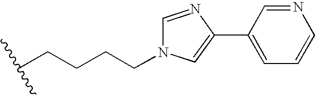 |
| 49 | 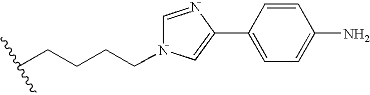 |
| 50 | 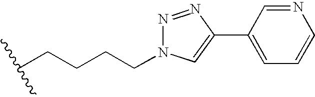 |
| 51 | 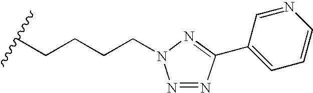 |
| 52 | 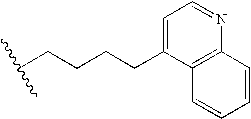 |
| 53 | 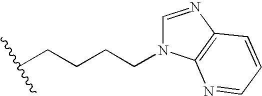 |
| 54 | 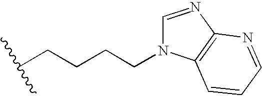 |
| 55 | 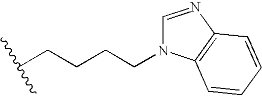 |

A further embodiment of the present invention includes pharmaceutical compositions comprising any single compound delineated herein, or a pharmaceutically acceptable salt, ester, or prodrug thereof, with a pharmaceutically acceptable carrier or excipient.

Yet another embodiment of the present invention is a pharmaceutical composition comprising a combination of two or more compounds delineated herein, or a pharmaceutically acceptable salt, ester, or prodrug thereof, with a pharmaceutically acceptable carrier or excipient.

Yet a further embodiment of the present invention is a pharmaceutical composition comprising any single compound delineated herein in combination with one or more antibiotics known in the art (such as penicillin, amoxicillin, azithromycin, erythromycin, ciprofloxacin, telithromycin, cethromycin, and the like), or a pharmaceutically acceptable salt, ester, or prodrug thereof, with a pharmaceutically acceptable carrier or excipient.

In addition, the present invention contemplates processes of making any compound delineated herein via any synthetic method delineated herein.

### DEFINITIONS

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system including, but not limited to, phenyl, naphthyl, tetrahydronaphthyl, indanyl, indenyl.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl.

In accordance with the invention, any of the aryls, substituted aryls, heteroaryls and substituted heteroaryls described herein, can be any aromatic group. Aromatic groups can be substituted or unsubstituted.

The term “C₁-C₆ alkyl,”, “C₁-C₈ alkyl,” or “C₁-C₁₂ alkyl,” as used herein, refer to saturated, straight- or branched-chain hydrocarbon radicals containing between one and eight, or one and twelve carbon atoms, respectively. Examples of C₁-C₈ alkyl radicals include, but are not limited to, methyl, ethyl, propyl, isopropyl, n-butyl, tert-butyl, neopentyl, n-hexyl, heptyl and octyl radicals; and examples of C₁-C₁₂ alkyl radicals include, but are not limited to, ethyl, propyl, isopropyl, n-hexyl, octyl, decyl, dodecyl radicals.

The term “C₂-C₆” or “C₂-C₈ alkenyl,” as used herein, refer to straight- or branched-chain hydrocarbon radicals containing from two to eight carbon atoms having at least one carbon-carbon double bond by the removal of a single hydrogen atom. Alkenyl groups include, but are not limited to, for example, ethenyl, propenyl, butenyl, 1-methyl-2-buten-1-yl, heptenyl, octenyl, and the like.

The term “C₂-C₆” or “C₂-C₈ alkynyl,” as used herein, refer to straight- or branched-chain hydrocarbon radicals containing from two to eight carbon atoms having at least one carbon-carbon triple bond by the removal of a single hydrogen atom. Representative alkynyl groups include, but are not limited to, for example, ethynyl, 1-propynyl, 1-butynyl, heptynyl, octynyl, and the like. The term “C₃-C₈-cycloalkyl”, or “C₃-C₁₂-cycloalkyl,” as used herein, refers to a monocyclic or polycyclic saturated carbocyclic ring compound. Examples of C₃-C₈-cycloalkyl include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo[2.2.1]heptyl, and bicyclo[2.2.2]octyl.

The term “C₃-C₈ cycloalkenyl”, or “C₃-C₁₂ cycloalkenyl” as used herein, refers to monocyclic or polycyclic carbocyclic ring compound having at least one carbon-carbon double bond. Examples of C₃-C₈ cycloalkenyl include, but not limited to, cyclopropenyl, cyclobutenyl, cyclopentenyl, cyclohexenyl, cycloheptenyl, cyclooctenyl, and the like; and examples of C₃-C₁₂ cycloalkenyl include, but not limited to, cyclopropenyl, cyclobutenyl, cyclopentenyl, cyclohexenyl, cycloheptenyl, cyclooctenyl, and the like.

It is understood that any alkyl, alkenyl, alkynyl and cycloalkyl moiety described herein can also be an aliphatic group, an alicyclic group or a heterocyclic group. An “aliphatic” group is a non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted.

The term “alicyclic,” as used herein, denotes a group derived from a monocyclic or bicyclic saturated carbocyclic ring compound. Examples include, but not limited to, cyclopropyl, cyclobutyl, cyclopentyl, cyclohexyl, bicyclo[2.2.1]heptyl, and bicyclo[2.2.2]octyl. Such alicyclic groups may be further substituted.

The terms “heterocyclic” or “heterocycloalkyl” can be used interchangeably and referred to a non-aromatic ring or a bi- or tri-cyclic group fused system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (iv) any of the above rings may be fused to an aromatic ring, and (v) the remaining ring atoms are carbon atoms which may be optionally oxo-substituted. Representative heterocyclic groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, and tetrahydrofuryl. Such heterocyclic groups may be further substituted.

The term “substituted” refers to substitution by independent replacement of one, two, or three or more of the hydrogen atoms thereon with substituents including, but not limited to, —F, —Cl, —Br, —I, —OH, protected hydroxy, —NO₂, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)-heteroaryl, —C(O)-heterocycloalkyl, —C(O)NH₂, —C(O)NH—C₁-C₁₂-alkyl, —C(O)NH—C₂-C₈-alkenyl, —C(O)NH—C₂-C₈-alkynyl, —C(O)NH—C₃-C₁₂-cycloalkyl, —C(O)NH-aryl, —C(O)NH-heteroaryl, —C(O)NH-heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —OC(O)NH₂, —OC(O)NH—C₁-C₁₂-alkyl, —OC(O)NH—C₂-C₈-alkenyl, —OC(O)NH—C₂-C₈-alkynyl, —OC(O)NH—C₃-C₁₂-cycloalkyl, —OC(O)NH-aryl, —OC(O)NH-heteroaryl, —OC(O)NH— heterocycloalkyl, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₈-alkenyl, —NHCO₂—C₂-C₈-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl —S(O)₂NH₂, —S(O)₂NH—C₁-C₁₂-alkyl, —S(O)₂NH—C₂-C₈-alkenyl, —S(O)₂NH—C₂-C₈-alkynyl, —S(O)₂NH—C₃-C₁₂-cycloalkyl, —S(O)₂NH-aryl, —S(O)₂NH-heteroaryl, —S(O)₂NH-heterocycloalkyl, —NHS(O)₂—C₁-C₁₂-alkyl, —NHS(O)₂—C₂-C₈-alkenyl, —NHS(O)₂—C₂-C₈-alkynyl, —NHS(O)₂—C₃-C₁₂-cycloalkyl, —NHS(O)₂-aryl, —NHS(O)₂-heteroaryl, —NHS(O)₂-heterocycloalkyl, —CH₂NH₂, —CH₂S(O)₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthiomethyl. It is understood that the aryls, heteroaryls, alkyls, and the like can be further substituted.

The term “monosaccharide” embraces radicals of cladinose, allose, altrose, arabinose, erythrose, erythrulose, fructose, D-fucitol, L-fucitol, fucosamine, fucose, galactosamine, D-galactosaminitol, galactose, glucosamine, glucosaminitol, glucose, glyceraldehyde, glycerol, glycerone, gulose, idose, lyxose, mannosamine, annose, psicose, quinovose, quinovosamine, rhamnitol, rhamnosamine, rhamnose, ribose, ribulose, sorbose, tagatose, tartaric acid, threose, xylose and xylulose. The monosaccharide may further be a deoxy sugar (alcoholic hydroxy group replaced by hydrogen), amino sugar (alcoholic hydroxy group replaced by amino group), a thio sugar (alcoholic hydroxy group replaced by thiol, or C═O replaced by C═S, or a ring oxygen of cyclic form replaced by sulfur), a seleno sugar, a telluro sugar, an aza sugar (ring carbon replaced by nitrogen), an imino sugar (ring oxygen replaced by nitrogen), a phosphano sugar (ring oxygen replaced with phosphorus), a phospha sugar (ring carbon replaced with phosphorus), a C-substituted monosaccharide (hydrogen at a non-terminal carbon atom replaced with carbon), an unsaturated monosaccharide, an alditol (carbonyl group replaced with CHOH group), aldonic acid (aldehydic group replaced by carboxy group), a ketoaldonic acid, a uronic acid, an aldaric acid, and so forth. Amino sugars include amino monosaccharides, preferably galactosamine, glucosamine, mannosamine, fucosamine, quinovosamine, neuraminic acid, muramic acid, lactosediamine, acosamine, bacillosamine, daunosamine, desosamine, forosamine, garosamine, kanosamine, kansosamine, mycaminose, mycosamine, perosamine, pneumosamine, purpurosamine, rhodosamine. It is understood that the monosaccharide and the like can be further substituted.

The terms “disaccharide”, “trisaccharide” and “polysaccharide” embrace radicals of abequose, amicetose, amylose, apiose, arcanose, ascarylose, ascorbic acid, boivinose, cellobiose, cellotriose, chacotriose, chalcose, colitose, cymarose, 2-deoxyribose, 2-deoxyglucose, diginose, digitalose, digitoxose, evalose, evemitrose, gentianose, gentiobiose, hamamelose, inulin, isolevoglucosenone, isomaltose, isomaltotriose, isopanose, kojibiose, lactose, lactosamine, lactosediamine, laminarabiose, levoglucosan, levoglucosenone, β-maltose, manninotriose, melezitose, melibiose, muramic acid, mycarose, mycinose, neuraminic acid, nigerose, nojirimycin, noviose, oleandrose, panose, paratose, planteose, primeverose, raffinose, rhodinose, rutinose, sarmentose, sedoheptulose, sedoheptulosan, solatriose, sophorose, stachyose, streptose, sucrose, α,α-trehalose, trehalosamine, turanose, tyvelose, umbelliferose and the like. Further, it is understood that the “disaccharide”, “trisaccharide” and “polysaccharide” and the like can be further substituted. Disaccharide also includes amino sugars and their derivatives, particularly, a mycaminose derivatized at the C-4′ position or a 4 deoxy-3-amino-glucose derivatized at the C-6′ position.

The term “trisaccharide” includes amino sugars and halo sugars, where halo sugars is saccharide group having at least one halogen substituent.

The term “halogen,” as used herein, refers to an atom selected from fluorine, chlorine, bromine and iodine.

The term “hydroxy activating group”, as used herein, refers to a labile chemical moiety which is known in the art to activate a hydroxyl group so that it will depart during synthetic procedures such as in a substitution or an elimination reaction. Examples of hydroxyl activating group include, but not limited to, mesylate, tosylate, triflate, p-nitrobenzoate, phosphonate and the like.

The term “activated hydroxy”, as used herein, refers to a hydroxy group activated with a hydroxyl activating group, as defined above, including mesylate, tosylate, triflate, p-nitrobenzoate, phosphonate groups, for example.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in T. H. Greene and P. G. M. Wuts, Protective Groups in Organic Synthesis, 3rd edition, John Wiley & Sons, New York (1999). Examples of hydroxyl protecting groups include benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl(trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxyl protecting groups for the present invention are acetyl(Ac or —C(O)CH₃), benzoyl(Bz or —C(O)C₆H₅), and trimethylsilyl(TMS or —Si(CH₃)₃).

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “hydroxy prodrug group”, as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in T. H. Greene and P. G. M. Wuts, Protective Groups in Organic Synthesis, 3rd edition, John Wiley & Sons, New York (1999). Examples of amino protecting groups include, but are not limited to, t-butoxycarbonyl, 9-fluorenylmethoxycarbonyl, benzyloxycarbonyl, and the like.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “protected amino,” as used herein, refers to an amino group protected with an amino protecting group as defined above.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

The term “protic solvent” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization.

As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject” as used herein refers to an animal. Preferably the animal is a mammal. More preferably the mammal is a human. A subject also refers to, for example, dogs, cats, horses, cows, pigs, guinea pigs, fish, birds and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)- , or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like.

Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to a compound of Formula I. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

The present invention also relates to solvates of the compounds of the invention, for example hydrates.

This invention also encompasses pharmaceutical compositions containing, and methods of treating bacterial infections through administering, pharmaceutically acceptable prodrugs of compounds of the formula I. For example, compounds of formula I having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula I. The amino acid residues include but are not limited to the 20 naturally occurring amino acids commonly designated by three letter symbols and also includes 4-hydroxyproline, hydroxyysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

As used herein, unless otherwise indicated, the term “bacterial infection(s)” or “protozoa infections”; includes, but is not limited to, bacterial infections and protozoa infections that occur in mammals, fish and birds as well as disorders related to bacterial infections and protozoa infections that may be treated or prevented by administering antibiotics such as the compounds of the present invention. Such bacterial infections and protozoa infections and disorders related to such infections include, but are not limited to, the following: pneumonia, otitis media, meningitis, sinusitus, bronchitis, tonsillitis, cystic fibrosis (CF) and mastoiditis related to infection by Streptococcus pneumoniae, Haemophilus influenzae, Moraxella catarrhalis, Staphylococcus aureus, Peptostreptococcus spp, or Pseudomonas spp.; pharyngitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), S. pyogenes, S. agalactiae, Streptococcal groups C-F (minute-colony streptococci), viridans streptococci, Corynebacterium spp., Clostridium spp., or Bartonella henselae; uncomplicated acute urinary tract infections related to infection by S. saprophyticus or Enterococcus spp.; urethritis and cervicitis; and sexually transmitted diseases related to infection by Chlamydia trachomatis, Haemophilus ducreyi, Treponema pallidum, Ureaplasma urealyticum, or Nesseria gonorrheae; toxin diseases related to infection by S. aureus (food poisoning and Toxic shock syndrome), or Groups A, S, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by C. trachomatis, N. gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; gastroenteritis related to infection by Campylobacter jejuni; intestinal protozoa related to infection by Cryptosporidium spp. odontogenic infection related to infection by viridans streptococci; persistent cough related to infection by Bordetella pertussis; gas gangrene related to infection by Clostridium perfringens or Bacteroides spp.; Skin infection by S. aureus, Propionibacterium acne; atherosclerosis related to infection by Helicobacter pylori or Chlamydia pneumoniae; or the like.

Bacterial infections and protozoa infections and disorders related to such infections that may be treated or prevented in animals include, but are not limited to, the following: bovine respiratory disease related to infection by P. haemolytica, P. multocida, Mycoplasma bovis, or Bordetella spp.; cow enteric disease related to infection by E. coli or protozoa (i.e., coccidia, cryptosporidia, etc.), dairy cow mastitis related to infection by S. aureus, S. uberis, S. agalactiae, S. dysgalactiae, Klebsiella spp., Corynebacterium, or Enterococcus spp.; swine respiratory disease related to infection by A. pleuropneumoniae., P. multocida, or Mycoplasma spp.; swine enteric disease related to infection by E. coli, Lawsonia intracellularis, Salmonella spp., or Serpulina hyodyisinteriae; cow footrot related to infection by Fusobacterium spp.; cow metritis related to infection by E. coli; cow hairy warts related to Infection by Fusobacterium necrophorum or Bacteroides nodosus; cow pink-eye related to infection by Moraxella bovis, cow premature abortion related to infection by protozoa (i.e. neosporium); urinary tract infection in dogs and cats related to infection by E. coli; skin and soft tissue infections in dogs and cats related to infection by S. epidermidis, S. intermedius, coagulase neg. Staphylococcus or P. multocida; and dental or mouth infections in dogs and oats related to infection by Alcaligenes spp., Bacteroides spp., Clostridium spp., Enterobacter spp., Eubacterium spp., Peptostreptococcus spp., Porphfyromonas spp., Campylobacter spp., Actinomyces spp., Erysipelothrix spp., Rhodococcus spp., Trypanosoma spp., Plasmodium spp., Babesia spp., Toxoplasma spp., Pneumocystis spp., Leishmania spp., and Trichomonas spp. or Prevotella spp. Other bacterial infections and protozoa infections and disorders related to such infections that may be treated or prevented in accord with the method of the present invention are referred to in J. P. Sanford at al., “The Sanford Guide To Antimicrobial Therapy,” 26th Edition, (Antimicrobial Therapy, Inc., 1996).

### Antibacterial Activity

Susceptibility tests can be used to quantitatively measure the in vitro activity of an antimicrobial agent against a given bacterial isolate. Compounds are tested for in vitro antibacterial activity by a micro-dilution method. Minimal Inhibitory Concentration (MIC) is determined in 96 well microtiter plates utilizing the appropriate broth medium for the observed bacterial isolates. Antimicrobial agents are serially diluted (2-fold) in DMSO to produce a concentration range from about 64 μg/ml to about 0.03 μg/ml. The diluted compounds (2 μl/well) are then transferred into sterile, uninoculated medium (0.2 mL) by use of a 96 fixed tip-pipetting station. The inoculum for each bacterial strain is standardized to approximately 5×10⁵ CFU/mL by optical comparison to a 0.5 McFarland turbidity standard. The plates are inoculated with 10 μl/well of adjusted bacterial inoculum. The 96 well plates are covered and incubated at 35+/−2° C. for 24 hours in ambient air environment. Following incubation, plate wells are visually examined by Optical Density measurement for the presence of growth (turbidity). The lowest concentration of an antimicrobial agent at which no visible growth occurs is defined as the MIC. The compounds of the invention generally demonstrated an MIC in the range from about 64 μg/ml to about 0.03 μg/ml.

All in vitro testing follows the guidelines described in the Approved Standards M7-A7 protocol, published by the Clinical Laboratory Standards Institute (CLSI).

The invention further provides compositions and methods of treating patients suffering from an inflammatory condition comprising administering to a patient in need thereof, a therapeutically effective amount of at least one compound of the invention. Specific examples of inflammatory conditions treatable according to the invention include, but are not limited to, scleritis; epi-scleritis; allergic conjunctivitis; pulmonary inflammatory diseases, particularly cystic fibrosis (CF), asthma, chronic obstructive pulmonary disease (COPD), allergic bronchopulmonary aspergillosis (ABPA), and sarcoidosis; procto-sigmoiditis; allergic rhinitis; arthritis; tendonitis; apthous stomatitis; and inflammatory bowel disease.

The invention further provides compositions and methods for i) prophylactic treatment of those patients susceptible to the symptoms CF including pulmonary infection and inflammation associated with CF, ii) treatment at the initial onset of symptoms of pulmonary infection and inflammation associated with CF, and iii) treatment of ongoing or relapsing symptoms of infection and inflammation associated with CF. In accordance with the invention a compound according to any one of compounds of the invention, is administered to a patient in need of treatment for CF, in amount sufficient to prevent, diminish or eradicate symptoms of CF including chronic pulmonary inflammation and infection.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al, and WO 98/43,650 by Montgomery, all of which are incorporated herein by reference). A discussion of pulmonary delivery of antibiotics is also found in U.S. Pat. No. 6,014,969, incorporated herein by reference.

According to the methods of treatment of the present invention, bacterial infections, cystic fibrosis and inflammatory conditions are treated or prevented in a patient such as a human or another animal by administering to the patient a therapeutically effective amount of a compound of the invention, in such amounts and for such time as is necessary to achieve the desired result.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment.

The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the formulae described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically exipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

Unless otherwise defined, all technical and scientific terms used herein are accorded the meaning commonly known to one of ordinary skill in the art. All publications, patents, published patent applications, and other references mentioned herein are hereby incorporated by reference in their entirety.

### Abbreviations

Abbreviations which may appear in the following synthetic schemes and examples are:
Ac for acetyl;
AcOH for acetic acid;
AIBN for azobisisobutyronitrile;
BSA for N,O-bis(trimethysilyl)acetamide;
BINAP for 2,2′-bis(diphenylphosphino)-1,1′-binaphthyl;
Boc₂O for di-tert-butyl-dicarbonate;
Boc for t-butoxycarbonyl;
Bpoc for 1-methyl-1-(4-biphenylyl)ethyl carbonyl;
Bz for benzoyl;
Bn for benzyl;
BocNHOH for tert-butyl N-hydroxycarbamate;
t-BuOK for potassium tert-butoxide;
Bu₃SnH for tributyltin hydride;
BOP for (benzotriazol-1-yloxy)tris(dimethylamino)phosphonium Hexafluorophosphate;
Brine for sodium chloride solution in water;
CDI for carbonyldiimidazole;
CH₂Cl₂ for dichloromethane;
CH₃ for methyl;
CH₃CN for acetonitrile;
Cs₂CO₃ for cesium carbonate;
CuCl for copper (I) chloride;
CuI for copper (I) iodide;
dba for dibenzylidene acetone;
dppb for diphenylphosphino butane;
DBU for 1,8-diazabicyclo[5.4.0]undec-7-ene;
DCC for N,N′-dicyclohexylcarbodiimide;
DEAD for diethylazodicarboxylate;
DIAD for diisopropyl azodicarboxylate;
DIPEA or (i-Pr)₂EtN for N,N,-diisopropylethyl amine;
Dess-Martin periodinane for 1,1,1-tris(acetyloxy)-1,1-dihydro-1,2-benziodoxol-3-(1H)-one;
DMAP for 4-dimethylaminopyridine;
DME for 1,2-dimethoxyethane;
DMF for N,N-dimethylformamide;
DMSO for dimethyl sulfoxide;
DPPA for diphenylphosphoryl azide;
EDC for N-(3-dimethylaminopropyl)-N′-ethylcarbodiimide;
EDC HCl for N-(3-dimethylaminopropyl)-N′-ethylcarbodiimide hydrochloride;
EtOAc for ethyl acetate;
EtOH for ethanol;
Et₂O for diethyl ether;
HATU for O-(7-azabenzotriazol-1-yl)-N,N,N′,N′,-tetramethyluronium Hexafluorophosphate;
HCl for hydrogen chloride;
HOBT for 1-hydroxybenzotriazole;
K₂CO₃ for potassium carbonate;
n-BuLi for n-butyl lithium;
i-BuLi for i-butyl lithium;
t-BuLi for t-butyl lithium;
PhLi for phenyl lithium;
LDA for lithium diisopropylamide;
TMEDA for N,N,N′,N′-tetramethylethylenediamine;
LiTMP for lithium 2,2,6,6-tetramethylpiperidinate;
MeOH for methanol;
Mg for magnesium;
MOM for methoxymethyl;
Ms for mesyl or —SO₂—CH₃;
Ms₂O for methanesulfonic anhydride or mesyl-anhydride;
NaN(TMS)₂ for sodium bis(trimethylsilyl)amide;
NaCl for sodium chloride;
NaH for sodium hydride;
NaHCO₃ for sodium bicarbonate or sodium hydrogen carbonate;
Na₂CO₃ sodium carbonate;
NaOH for sodium hydroxide;
Na₂SO₄ for sodium sulfate;
NaHSO₃ for sodium bisulfite or sodium hydrogen sulfite;
Na₂S₂O₃ for sodium thiosulfate;
NH₂NH₂ for hydrazine;
NH₄HCO₃ for ammonium bicarbonate;
NH₄Cl for ammonium chloride;
NMMO for N-methylmorpholine N-oxide;
NaIO₄ for sodium periodate;
Ni for nickel;
OH for hydroxyl;
OsO₄ for osmium tetroxide;
TEA or Et₃N for triethylamine;
TFA for trifluoroacetic acid;
THF for tetrahydrofuran;
TPP or PPh₃ for triphenylphosphine;
Troc for 2,2,2-trichloroethyl carbonyl;
Ts for tosyl or —SO₂—C₆H₄CH₃;
Ts₂O for tolylsulfonic anhydride or tosyl-anhydride;
TsOH for p-tolylsulfonic acid;
Pd for palladium;
Ph for phenyl;
POPd for dihydrogen dichlorobis(di-tert-butylphosphinito-κP)palladate(II);
Pd₂(dba)₃ for tris(dibenzylideneacetone) dipalladium (0);
Pd(PPh₃)₄ for tetrakis(triphenylphosphine)palladium (0);
PdCl₂(Ph₃P)₂ for trans-dichlorobis(triphenylphosphine)palladium (II);
Pt for platinum;
Rh for rhodium;
Ru for ruthenium;
TBS for tert-butyl dimethylsilyl; or
TMS for trimethylsilyl;
TMSCl for trimethylsilyl chloride.

### Synthetic Methods

The compounds and processes of the present invention will be better understood in connection with the following synthetic schemes that illustrate the methods by which the compounds of the invention may be prepared, which are intended as an illustration only and not to limit the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims. The compounds of the present invention can be prepared by methods which are well known in the art such as processes described by Baker et al. J. Org. Chem. 1988, 53, 2340-2345; Elliott et al. J. Med. Chem. 1988, 41, 1651-1659; Ma et al. J. Med. Chem. 2001, 44, 4137-4156, and Or et al. U.S. Pat. No. 6,075,011.

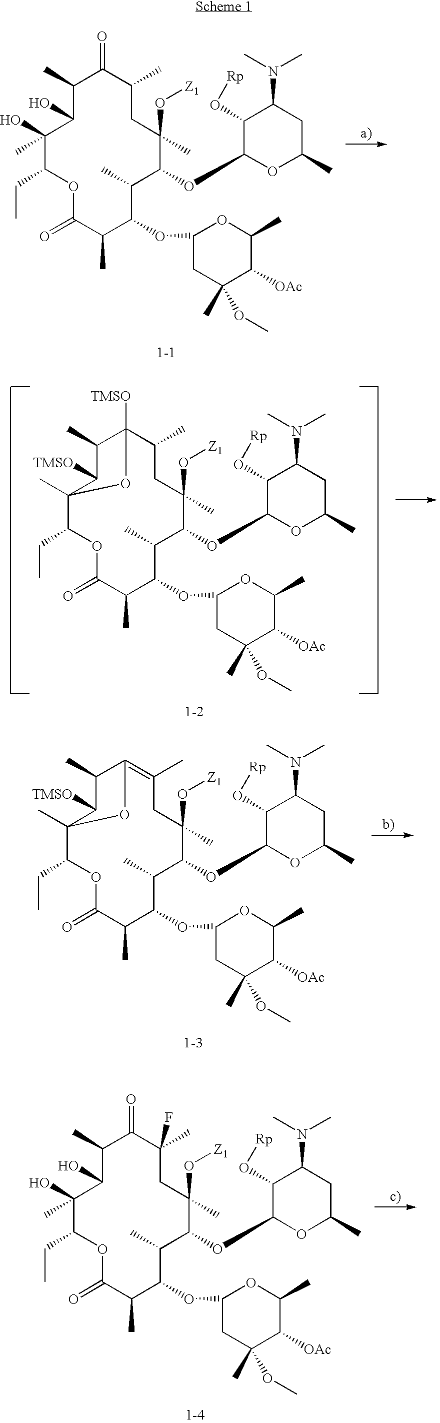

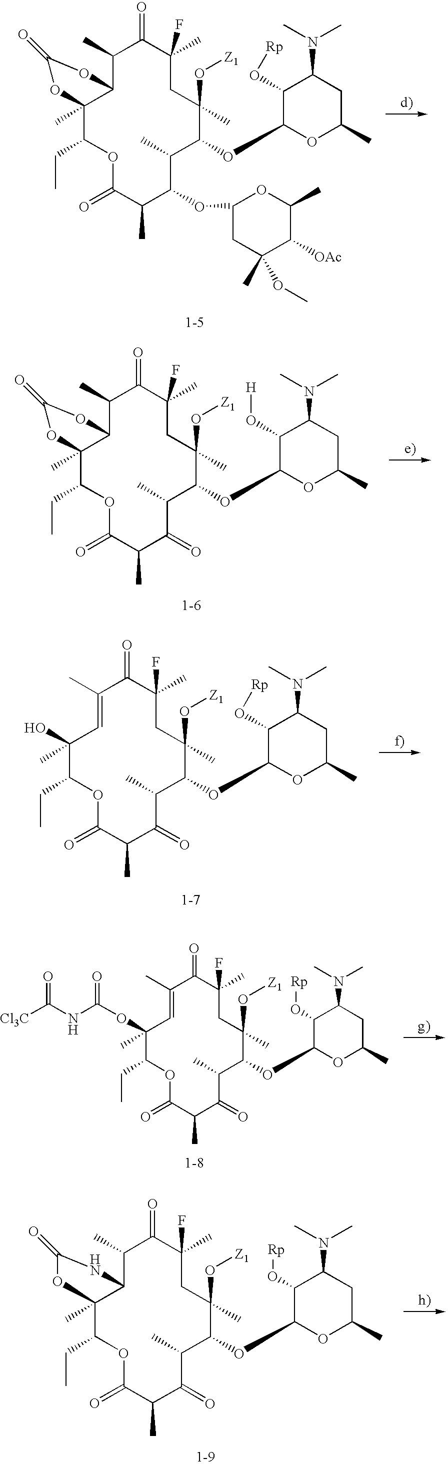

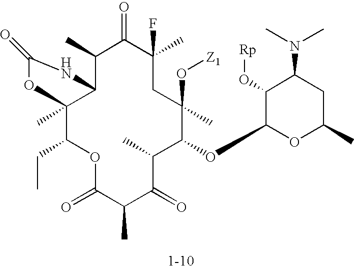

Reagents and Conditions: a) TMSOTf, 2,6-di-tert-butyl-pyridine, CH₂Cl₂, rt then reflux; b) Selectfluor, MeSO₃H, 2% H₂O in MeCN, 10° C.; c) Cl₃COCOCl, pyridine, CH₂Cl₂, 0° C.; d) (i) 1N—HCl, 70° C.; (ii) Dess-Martin, acetic acid, 0° C. then room temperature; (iii) methanol, 30° C.; e) DBU(1.5 eq)/Toluene; 50° C., 3 hrs; f) Cl₃C(O)NCO, CH₂Cl₂, 0° C., 3 hrs; g) IPA/RT to 50˜60° C., 2 days; h) t-BuOK(2 eq), THF-t-BuOH, 0° C. to RT or alternatively NH₄OH/MeCN/rt.

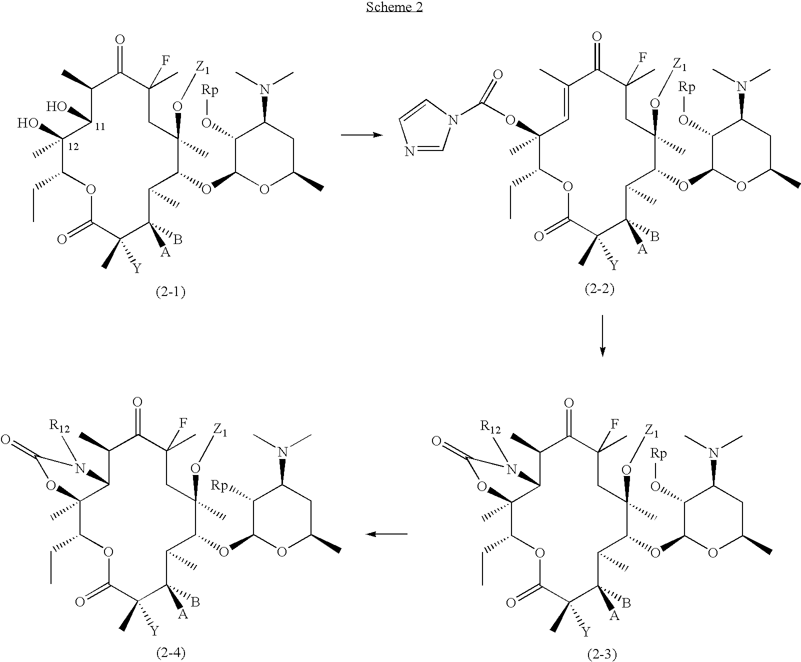

A scheme for the preparation of C11-C12 cyclic carbamates of formula (2-4) is shown is Scheme 2. Metallation of formula (2-1) with KHMDS at low temperature (e.g. 0° C.) and a subsequent reaction with carbonyl diimidazole (CDI) provide imidazolocarbonyl derivative (2-2), which is then reacted with ammonia or R₁₂ MH₂, where M and R₁₂ are previously defined, in acetonitrile at elevated temperature to give (2-3). The product is the N-substituted or unsubstituted oxazolidinone (2-3). In the event that the resulting product has an unnatural C 10-stereochemisty, it can be equilibrated to the desired natural stereochemisty by treatment with an amine or alkoxide base. The target compounds are available by removal of the 2′-hydroxyl protection group in the sugar moiety by conditions known in the art.

Alternatively, compounds of formula (2-2) can be obtained via a stepwise by treating compounds of formula (2-1) with ethylene carbonate and triethyl amine at elevated temperature, followed by reacting C12-hydroxyl moiety with CDI in the present of a base or any phosgene equivalents.

Scheme 3 illustrates an alternative method of preparing compounds of the invention via flurithromycin. R¹⁴ is hydrogen, aryl, substituted aryl, heteroaryl, or substituted heteroaryl.

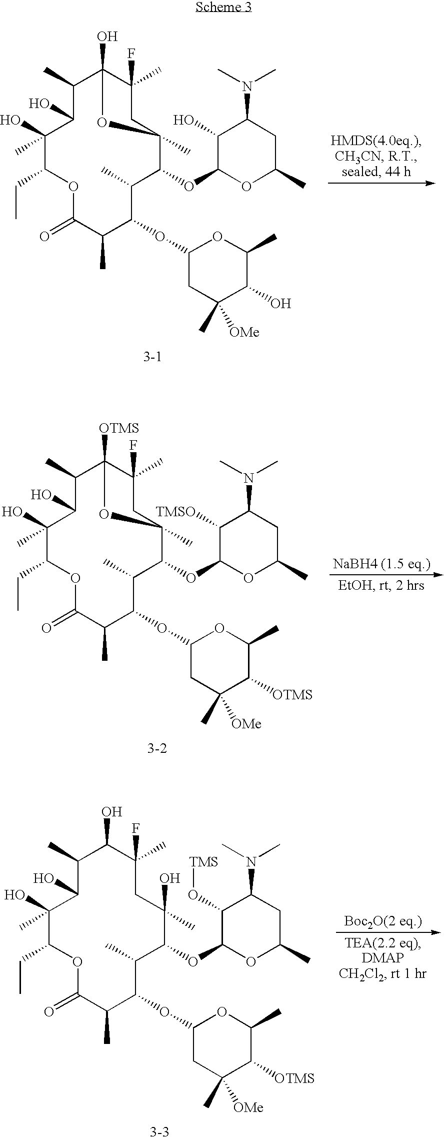

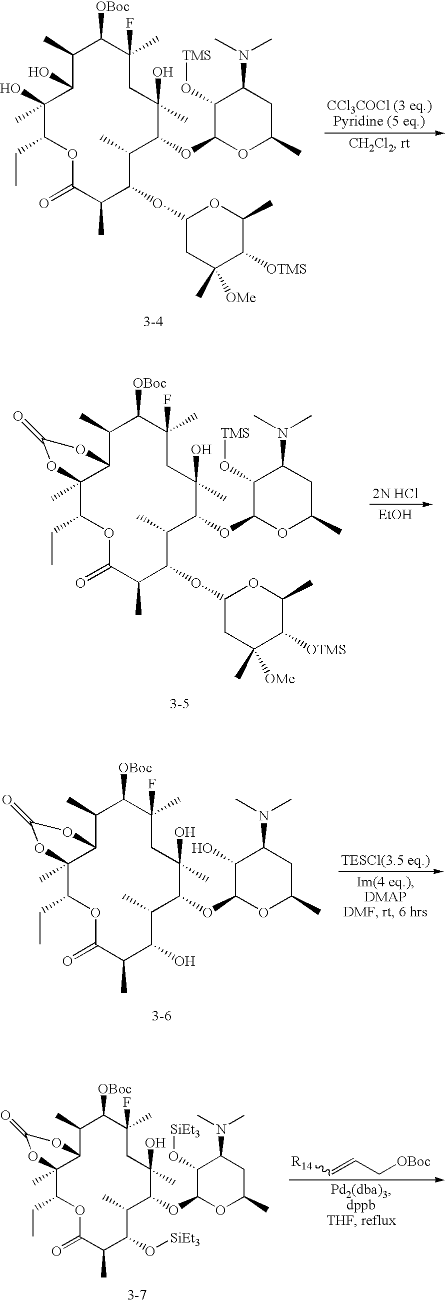

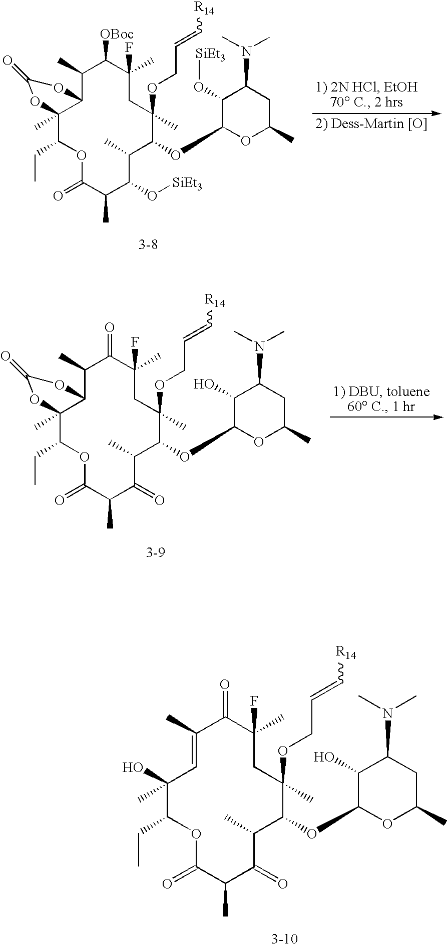

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

### EXAMPLES

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

### Example 1

### Compound of Formula II, Wherein U and V Taken Together is Oxo, G is Hydroxyl W and Y are Hydrogen B is

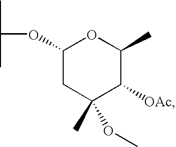

### A is Hydrogen C10 and C11 is a Single Bond Rp is Ac Z₁ is Allyl and R₄ and R₅ Are Each Methyl

### Step 1a:

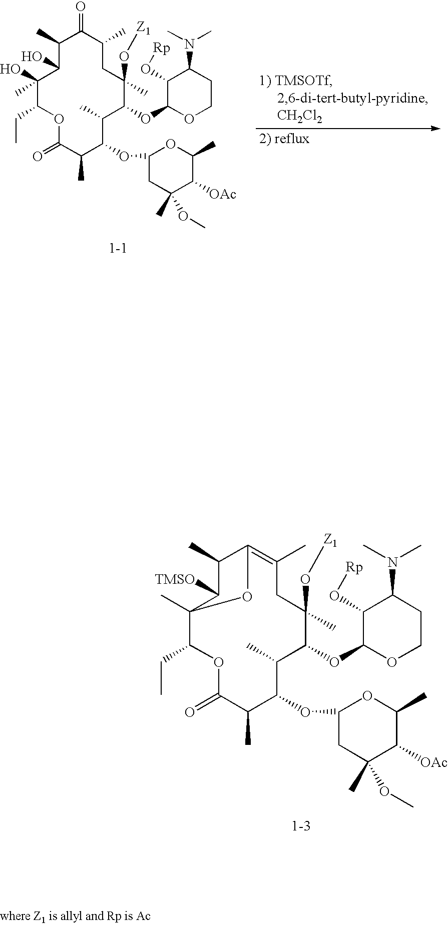

To a mixture of 6-O-allyl 2′, 4″-diacetate erythromycin (1-1, Z₁=allyl) (5.148 g, 6.0 mmol) and 2,6-di-tert-butyl-pyridine (4.24 mL, 18.9 mmol) in anhydrous methylene chloride (60 mL) was dropwise added TMSOTf (3.25 mL, 18.0 mmol) at room temperature and stirred for 1 hr at room temperature (The formation of 1-2 was identified by mass spectrum; MS (ESI) [1002 (M+H⁺)]. Then, it was heated at 40° C. for 40 hr and refluxed for 24 hr. It was dilute with methylene chloride (150 mL), washed with sat. sodium bicarbonate (50 mL) and 1M-sodium hydroxide solution (1 mL). The organic layer was washed with water (50 mL) and brine (30 mL). After drying with anhydrous sodium sulfate and evaporation of solvent, the residue was purified by column chromatography using 0-25% acetone in hexane to give a white foam of the titled compound (3.62 g, 66% over 2 steps). MS (ESI) m/z=912 (M+H⁺).

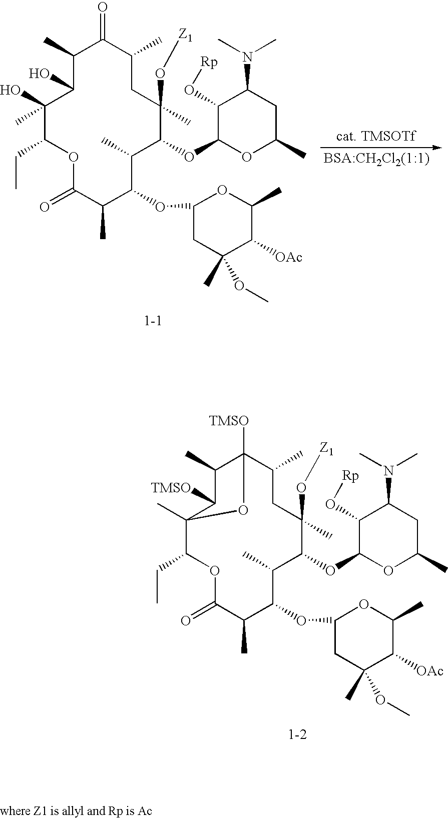

Alternative preparation of hemiketal 1-2: To a mixture of 6-O-allyl 2′, 4″-diacetate erythromycin (1-1, Z₁=allyl) (0.215 g, 0.25 mmol) in anhydrous methylene chloride and bis-trimethylsilyl acetamide(1:1, 1.25 mL) was dropwise added TMSOTf (4.5 μL, 25 μmol) at room temperature and stirred for 6.5 hr. It was diluted with methylene chloride (10 mL) and poured into sat. NaHCO₃ solution (10 mL) and stirred vigorously for 1.5 hr. The organic layer was isolated and washed with water (5 mL) and brine (5 mL). The organic layer was dried with anhydrous sodium sulfate and evaporated to give the titled compound as quantitative yield. MS (ESI) m/z=1002 (M+H⁺).

### Step 1b:

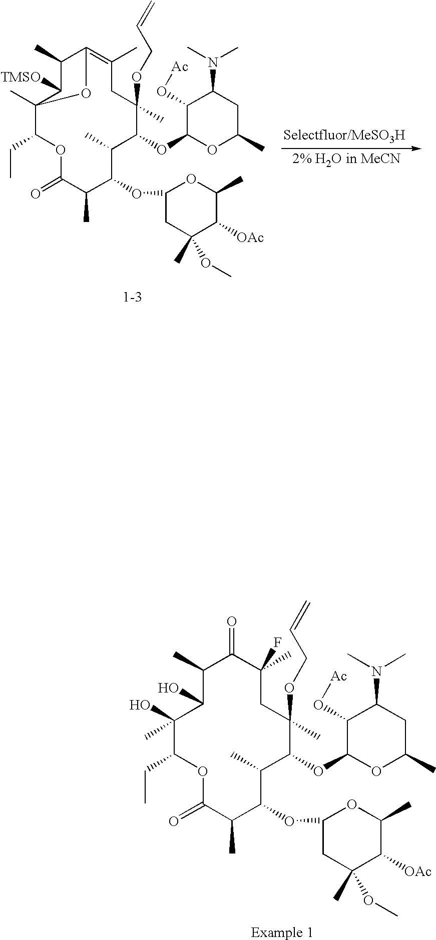

To a mixture of compound from Step 1a (5.068 g, 5.557 mmol) in 2% water in acetonitrile (222 mL) was added methanesulfonic acid (0.4 mL, 6.11 mmol) at 10° C. and stirred for 4 min. Then, selectfluor (2.067 g, 5.83 mmol) was added at 10° C. and stirred for 1 hr. Selectfluor (138 mg) was added stirred for 20 min. Ethyl vinyl ether (5 mL) was added to the reaction and stirred for 30 min. After evaporation in vacuo, it was diluted with methylene chloride (500 mL), washed with sat. NaHCO₃ solution, water and brine. After drying with anhydrous sodium sulfate and evaporation of solvent, , it was filtered and evaporated in vacuo. The residue was purified by column chromatography using 0-40% acetone in hexane to give a white foam of the titled compound (4.235 g, 87%). MS (ESI) m/z=876 (M+H⁺).

### Example 2

### Compound of Formula II, Wherein U and V Taken Together is Oxo, G and W Taken Together is

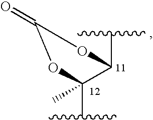

### A and Y are Hydrogen B is

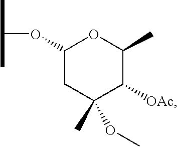

### C10 and C11 is a Single Bond, Rp is Ac, Z₁ is Allyl and R₄ and R₅ are Each Methyl

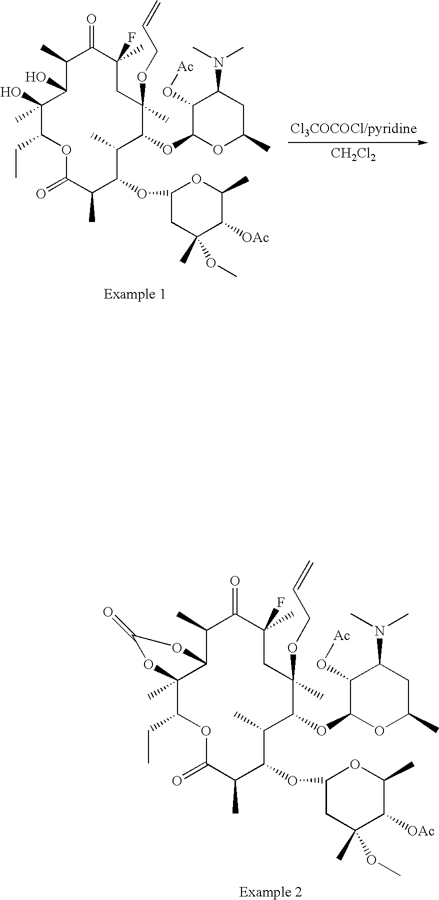

To a solution of compound from Example 1 (120 mg, 0.137 mmol) and pyridine (55 μL) in abs. methylene chloride (1.6 mL) was added trichloromethyl chloroformate (50 μL, 0.41 mmol) at 0° C. and stirred for 3 hrs. Then, it was poured into cold saturated NaHCO₃ solution (5 mL) and stirred vigorously for 5 min. The organic layer was isolated and diluted with methylene chloride (10 mL) and washed with Brine solution. After drying with anhydrous sodium sulfate and evaporation of solvent, it was filtered and evaporated in vacuo. The residue was purified by column chromatography using 0-40% acetone in hexane to give a white foam of the titled compound (79 mg, 64%). MS (ESI) m/z=902 (M+H⁺).

### Example 3

### Compound of Formula II Wherein U and V Taken Together is Oxo, G and W taken

### Together is

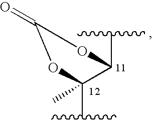

### Y is Hydrogen, A and B Taken Together is Oxo, C10 and C11 is a Single Bond, Rp is Ac, Z₁ is Allyl and R₄ and R₅ are Each Methyl.

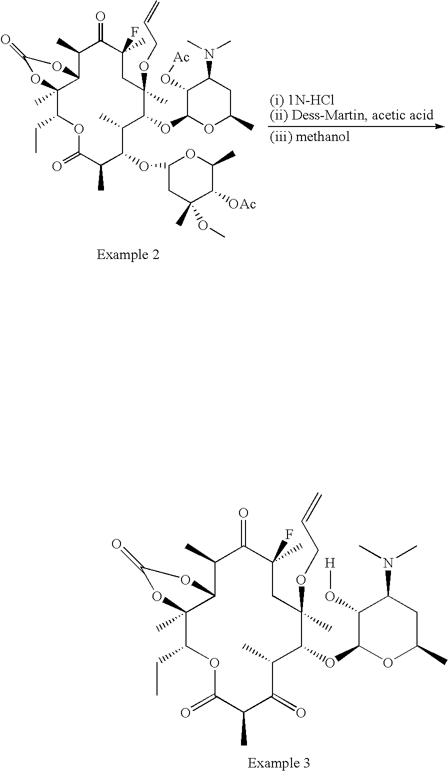

A mixture of compound from Example 2 (79 mg) in 1N—HCl (2 mL) was heated at 70° C. for 2 hr. After cooling to room temperature, the reaction was extracted with methyl tert-butyl ether (3×5 mL). Aquous layer was basified with cold saturated NaHCO₃ solution and extracted with methylene chloride (3×5 mL), washed with water (5 mL) and Brine solution (5 mL). After drying with anhydrous sodium sulfate and evaporation of solvent, it was filtered and evaporated in vacuo. The residue was dissolved in methylene chloride (970 μL). Acetic acid (8 μL) and Dess-Martin periodinane (43 mg, 0.1 mmol) were successively added at 0° C. and stirred at room temperature for 1 hr. The reaction was diluted with methylene chloride (5 mL), washed with saturated NaHCO₃ and 1M-NaOH aqueous solution. The organic layer was washed with water and Brine. After drying with anhydrous sodium sulfate and evaporation of solvent, it was filtered and evaporated in vacuo. The residue was purified by short column chromatography using 10-40% acetone in hexane to give Diketone. MS (ESI) m/z=700 (M+H⁺). Diketone was diluted with methanol (3 mL) and heated at 30° C. for 12 hrs. After evaporation, it was purified by column chromatography using 0-6% 0.5M-NH₃ in methanol in methylene chloride) to give the titled compound as a white solid (27 mg, 47% over 3 steps). MS (ESI) m/z=658 (M+H⁺).

### Example 4

### Compound of Formula II, Wherein U and V Taken Together is Oxo, G and W Taken Together is

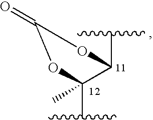

### Y is Hydrogen C10 and C₁₁ is a Double Bond, A and B Taken Together is Oxo, Rp is Ac, Z₁ is

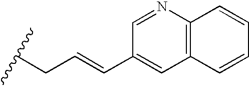

### and R₄ and R₅ are Each Methyl.

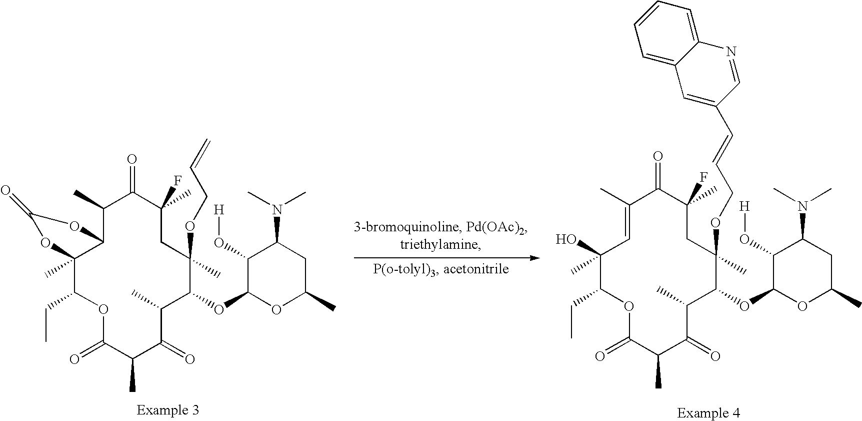

A mixture of compound from Example 3 (27 mg, 0.04 mmol), 3-bromoquinoline (11 μL, 0.08 mmol), P(o-tolyl)₃ (5 mg, 0.016 mmol) and triethylamine (11.4 μL, 0.08 mmol) in acetonitrile (200 μL) was degassed and Pd(OAc)₂ (1.8 mg, 0.008 mmol) was added. Then, it was heated in microwave at 130° C. for 30 min. The reaction was diluted with methylene chloride (5 mL), washed with saturated NaHCO₃, water and Brine solution. After drying with anhydrous sodium sulfate and evaporation of solvent, it was filtered and evaporated in vacuo. The residue was purified by column chromatography using 0-4% 0.5M-NH₃ in methanol in methylene chloride) to give a mixtures of the product and C6-enol. Example 4 was obtained from the purification using reverse phase HPLC (30-80% NH₄HCO₃ in acetonitrile as a mobile phase). MS (ESI) m/z=741 (M+H⁺).

### Example 5

### Compound of Formula II, Wherein U and V Taken Together is Oxo, G and W Taken Together is

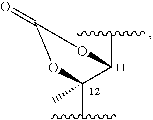

### Y is Hydrogen, B is

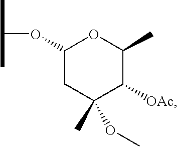

### A is Hydrogen C10 and C11 is a Single Bond, Rp is Bz, Z₁ is Allyl and R₄ and R₅ are Each Methyl.

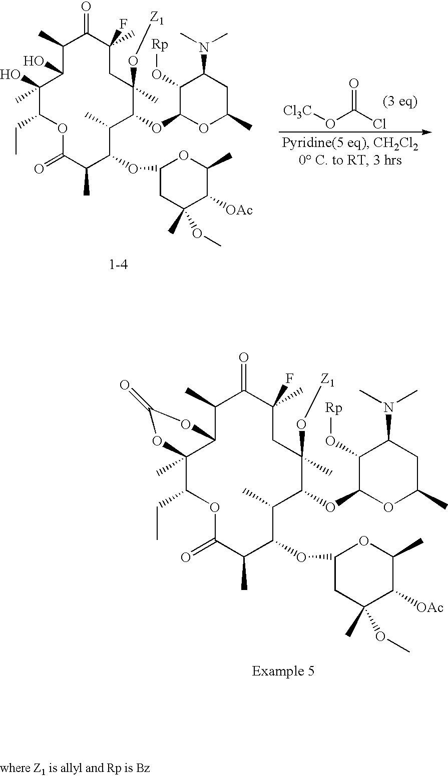

To a solution of compound I-4 of Scheme 1 (600 mg, 0.64 mmol) in CH₂Cl₂ (7.1 mL) was added Pyridine (0.26 mL, 3.2 mmol), Trichloromethyl chloroformate (0.23 mL, 1.92 mmol) at 0° C. and warmed to room temperature. The reaction mixture was stirred for 3 hours at room temperature until starting material was disappeared. The resulting reaction mixture was poured to pre-cooled saturated sodium bicarbonate solution (50 ml), stirred for 5 min and diluted with CH₂Cl₂(50 ml). The organic layers were separated and the resulting aqueous layer was extracted with CH₂Cl₂ (50 mL×3). The combine organic layers were dried (Na₂SO₄) and filtered. The filtrate was concentrated and purified by combi-flash chromatography (silica gel, 100% Hexanes to 30% Acetone/Hexanes) giving 540 mg of the titled compound. MS (ESI) m/z=964.7 (M+H)⁺.

### Example 6

### Compound of Formula II, Wherein U and V Taken Together is Oxo, G and W Taken Together is

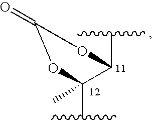

### Y is Hydrogen, B is OH, A is Hydrogen, C10 and C11 is a Single Bond, Rp is Bz, Z₁ is Allyl and R₄ and R₅ are Each Methyl.

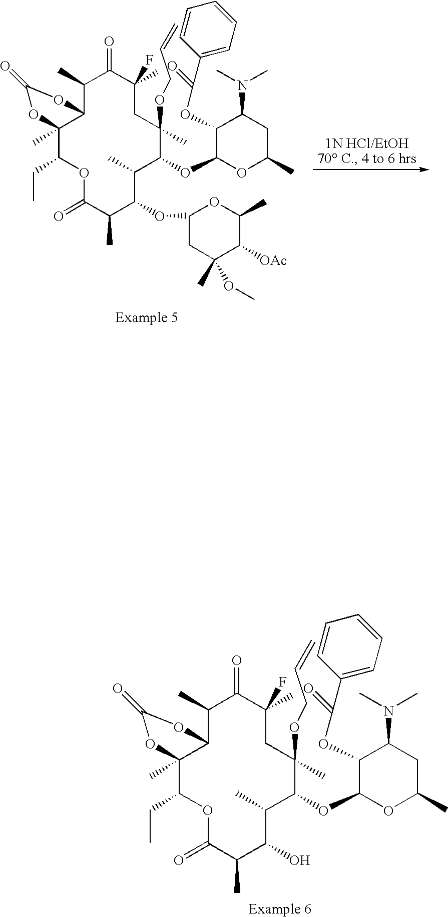

To a solution of compound from Example 5 (200 mg, 0.207 mmol) in Ethanol (2 mL), was added 1N HCl (2 mL) and stirred for 4 hrs at 60-70° C. The resulting mixture was cooled to 0° C. added H₂O (8 mL) the extracted with diethyl ether (20 mL×2, removed cleaved sugar). The aqueous layer was diluted with CH₂Cl₂ (20 mL), neutralized with saturated NaHCO₃ solution, separated organic layers then the resulting aqueous layers were extracted with the CH₂Cl₂ (20 mL×2). The combined organic layers were concentrated giving 159 mg of the title compound. Without any further purification used for the next step. MS (ESI) m/z=764.5 (M+H)⁺.

### Example 7

### Compound of Formula II, Wherein U and V Taken Together is Oxo, G and W Taken Together is

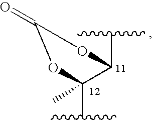

### Y is Hydrogen, A and B Taken Together is Oxo, C10 and C₁₁ is a Single Bond, Rp is Bz, Z₁ is Allyl and R₄ and R₅ are Each Methyl.

A mixture of compound from Example 6 (159 mg, 0.208 mmol), HOAc (0.012 mL) and Dess-Martin periodinane (132 mg, 0.312 mmol) in CH₂Cl₂ (4 mL) was stirred at room temperature for 2 hours. The reaction mixture was quenched with saturated NaHCO₃ solution (5 mL) and resulting mixture was washed with 0.5 N NaOH (10 mL). The organic phase was dried (Na₂SO₄) and filtered. The filtrate was concentrated and purified by flash column chromatography (silica gel, 10% Acetone/Hexanes to 30% Acetone/Hexanes) giving 85 mg of the titled compound. MS (ESI) m/z=762.5 (M+H)⁺.

### Example 8

### Compound of Formula II Wherein U and V Taken Together is Oxo, G is Hydroxyl W and Y are Hydrogen C₁₀ and C11 is a Double Bond, A and B Taken Together is Oxo, Rp is Bz, Z₁ is Allyl and R₄ and R₅ are Each Methyl

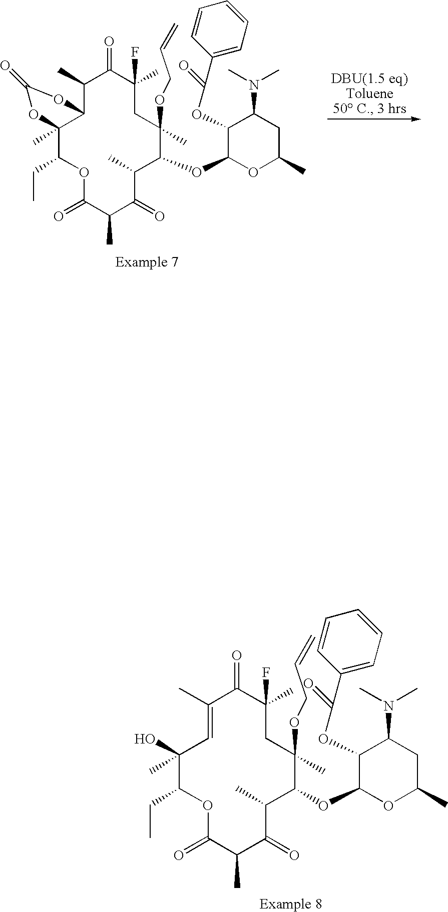

A mixture of compound from Example 7 (85 mg, 0.11 mmol), DBU (0.033 ml, 0.22 mmol) in Toluene (2 mL) was stirred at 50° C. for 3 hours. The reaction mixture was cooled room temperature diluted with EtOAc (10 ml) and washed with saturated NaHCO₃ (5 mL×2) solution. The separated organic phase was dried (Na₂SO₄) and filtered. The filtrate was concentrated and purified by flash chromatography (silica gel, 10% Acetone/Hexanes to 20% Acetone/Hexanes) giving 50 mg of the titled compound. MS (ESI) m/z=718.3 (M+H)⁺.

### Example 9

### Compound of Formula II Wherein U and V Taken Together is Oxo, G is Hydroxyl W is

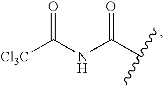

### Y is Hydrogen, C₁₀ and C11 is a Double Bond, A and B Taken Together is Oxo, Rp is Bz, Z₁ is Allyl and R₄ and R₅ are Each Methyl.

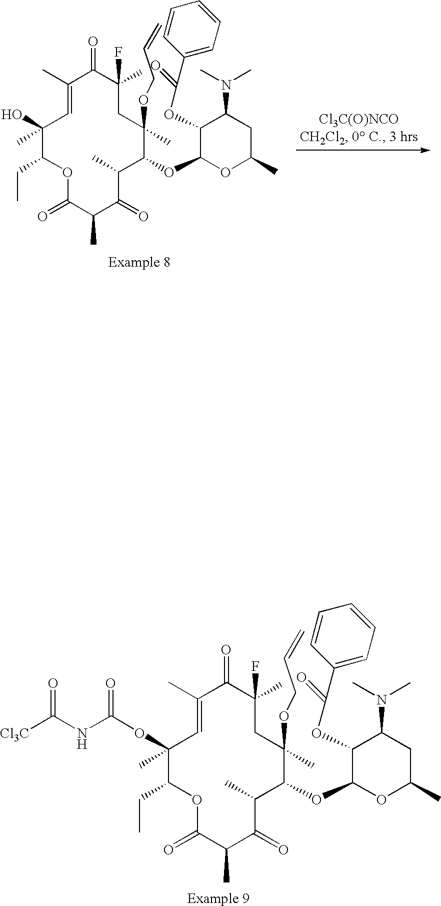

To a solution of compound from Example 8 (50 g, 0.07 mmol) in CH₂Cl₂ (1 mL), which was cooled to 0° C., added Trichloroacetyl isocyanate (0.017 mL, 0.139 mmol) as slowly drop wised. The resulting mixture was stirred at 0° C. for 3 hours. The reaction mixture was added 5 mL of IPA, stirred for 5 min and concentrated all the solvent under reduced pressure to give the titled compound 100 mg. Without any further purification used for the next step. MS (ESI) m/z=905.5, 907.5 (M+H)⁺.

### Example 10

### Compound of Formula II Wherein U and V Taken Together is Oxo, G and W Taken Together is

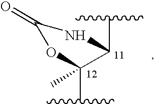

### Y is Hydrogen, A and B Taken Together is Oxo, C10 and C11 is a Single Bond, Rp is Bz, Z₁ is Allyl and R₄ and R₅ are Each Methyl (the C10 S-Isomer).

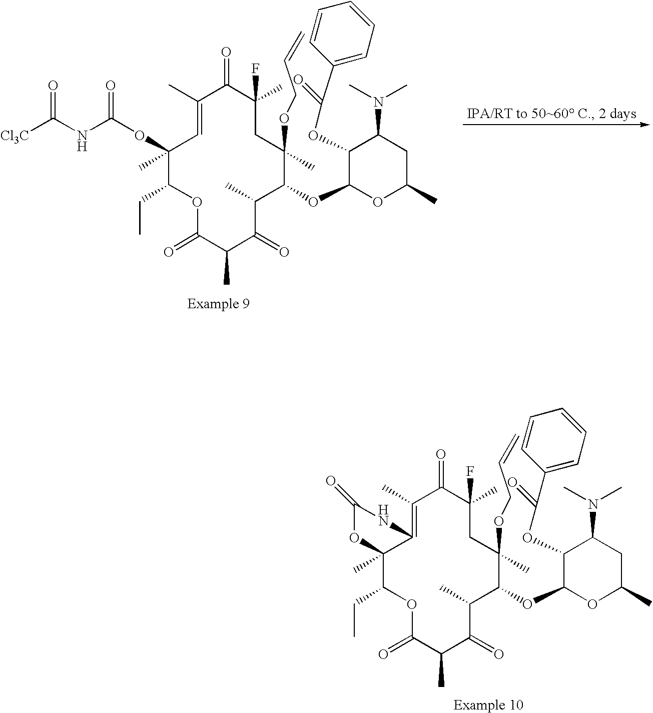

A solution of compound from Example 9 (100 mg, 0.07 mmol) in IPA (3 mL) was stirred at 55-60° C. for 48 hours then evaporated all the solvent. The resulting mixture was dissolved in Ethyl acetate (10 mL) washed with saturated NaHCO₃ (5 mL×2) and Brine solution (5 mL). The organic layers were dried (Na₂SO₄), and removed all the solvent under reduced pressure and purified by flash chromatography (silica gel, 10% Acetone/Hexanes to 30% Acetone/Hexanes) giving 20 mg of the titled compound. MS (ESI) m/z=761.6 (M+H)⁺.

### Example 11

### Compound of Formula II, Wherein U and V Taken Together is Oxo, G and W Taken Together is

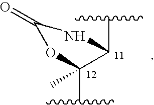

### Y is Hydrogen, A and B Taken Together is Oxo, C10 and C11 is a Single Bond, Rp is Bz, Z₁ is Allyl and R₄ and R₅ are Each Methyl (the C10 R-isomer).

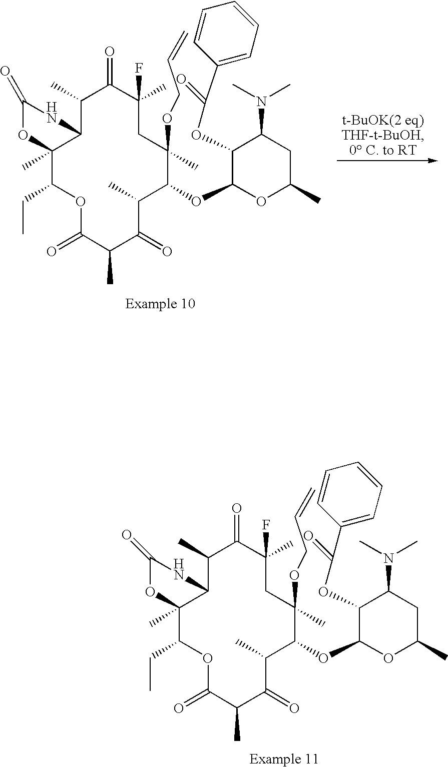

A solution of compound from Example 10 (10 mg, 0.013 mmol) in THF-^{t}BuOH(1:1 ratio, 1 mL) which was cooled to 0° C. and added K^{t}BuO(0.013 mL, 1N in THF solution) and warmed to room temperature. The resulting solution was stirred until SM was disappeared. The resulting mixture was dissolved in Ethyl acetate (10 mL) washed with saturated NaHCO₃ (2 mL×2) and Brine solution (5 mL). The organic layers were dried (Na₂SO₄), and removed all the solvent under reduced pressure The resulting mixture was purified by flash chromatography (silica gel, 10% Acetone/Hexanes to 30% Acetone/Hexanes) giving 2.5 mg of the title compound. MS (ESI) m/z=761.6 (M+H)⁺.

### Example 12

### Compound of Formula II Wherein U and V Taken Together is Oxo, G and W Taken Together is

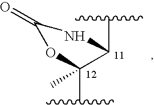

### Y is Hydrogen, A and B Taken Together is Oxo, C10 and C11 is a Single Bond, Rp is Hydrogen, Z₁ is Allyl and R₄ and R₅ are Each Methyl (the C10 S-Isomer).

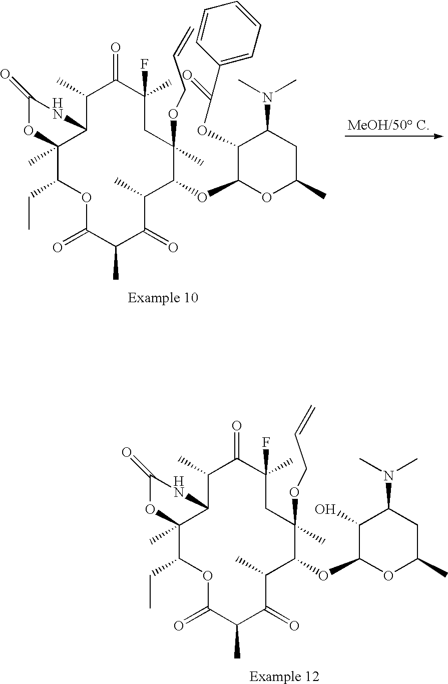

To a solution of compound Example 10 in 5 mL of Methanol, which was stirred at 50° C. until starting material was disappeared. The excess of solvent was evaporated under the reduced pressure to give 2.8 mg of the title compound. MS (ESI) m/z=657.4 (M+H)⁺.

Although the invention has been described with respect to various preferred embodiments, it is not intended to be limited thereto, but rather those skilled in the art will recognize that variations and modifications may be made therein which are within the spirit of the invention and the scope of the appended claims.

What is claimed is:

1. Compounds represented by formula (I): 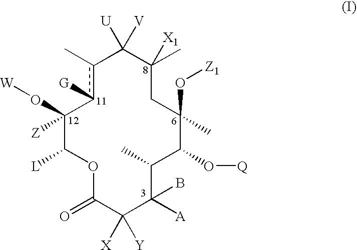
or the racemates, enantiomers, diastereomers, geometric isomers, tautomers, solvates, pharmaceutically acceptable salts, esters and prodrugs thereof,
wherein one of U and V is hydrogen or hydroxy and the other is selected from:
(a) hydrogen;
(b) —OR₁; where R₁ is independently selected from the group consisting of:
(i) hydrogen;
(ii) aryl; substituted aryl; heteroaryl; substituted heteroaryl;
(iii) —R₂, where R₂ is substituted or unsubstituted —C₁-C₈ alkyl, —C₂-C₈ alkenyl, or —C₂-C₈ alkynyl each containing 0, 1, 2, or 3 heteroatoms selected from O, S or N; and
(iv) —R₃, where R₃ is substituted and unsubstituted —C₃-C₁₂ cycloalkyl each containing 0, 1, 2, or 3 heteroatoms selected from O, S or N;
(c) —R₂;
(d) —OC(O)NHR₁;
(e) —OC(O)OR₁;
(f) —NR₄R₅; where R₄ and R₅ are each independently selected from R₁; alternatively, R₄ and R₅ taken together with the nitrogen atom to which they are connected form a 3- to 10-membered ring which may optionally contain one or more heterofunctions selected from the group consisting of: —O—, —NH—, —N(C₁-C₈-alkyl)-, —N(R₆)—, —S(O)ₙ—, wherein n=0, 1 or 2, and R₆ is selected from aryl; substituted aryl; heteroaryl; and substituted heteroaryl;
(g) —NHC(O)R₁;
(h) —NHS(O)₂R₁;
(i) —NHC(O)OR₁; and
(j) —NHC(O)NHR₁;
alternatively, U and V taken together with the carbon atom to which they are attached are selected from:
(a) C(O);
(b) C═N-J-R₁, where J is absent, O, C(O), S(O)₂, NH, NHC(O), NHC(O)NH or NHS(O)₂;
(c) C═CH-J-R₇; and wherein R₇ is independently selected from halogen and R₁;
(d) substituted or unsubstituted, and saturated or unsaturated 5- to 10-membered heterocyclic;
X₁ is halogen;
Z₁ is independently selected from:
(a) aryl; substituted aryl; heteroaryl; substituted heteroaryl;
(b) substituted or unsubstituted —C₁-C₈ alkyl, —C₂-C₈ alkenyl, or —C₂-C₈ alkynyl each containing 0, 1, 2, or 3 heteroatoms selected from O, S or N; and
(c) substituted or unsubstituted —C₃-C₁₂ cycloalkyl, or —C₃-C₁₂ cycloalkenyl each containing 0, 1, 2, or 3 heteroatoms selected from O, S or N;
or Z₁ and either A or B can be taken together to form cyclic structure 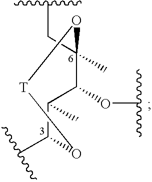
wherein T is
(a) —R₈—, where R₈ is substituted or unsubstituted —C₁-C₈ alkylene-, —C₂-C₈ alkenylene- or —C₂-C₈ alkynylene-, containing 0, 1, 2, or 3 heteroatoms selected from O, S or N;
(b) —R₈—C(O)—R₉—, where R₉ is independently selected from R₈;
(c) —R₈—(C═N-E-R₁)—R₉—, where E is absent, O, NH, NHC(O), NHC(O)NH or NHS(O)₂;
(d) —R₈—[C(OR₁₀)(OR₁₁)]—R₉—, where R₁₀ and R₁₁ are selected from the group consisting of C₁-C₁₂ alkyl, aryl or substituted aryl; or R₁₀ and R₁₁ taken together is —(CRₐR_{b})ᵣ—, where r is 2 or 3; Rₐ and R_{b} are independently selected from hydrogen, aryl, and R₂;
(e) —R₈—[C(SR₁₀)(SR₁₁)]—R₉—; or
(f) —R₈—(C═CH—R₁)—R₉—;
G is selected from the group consisting of:
a) hydrogen;
b) hydroxy;
c) —O—R₂; and
d) —O—R₆;
W is selected from:
(a) hydrogen;
(b) —R₂;
(c) —C(O)R₁;
(d) —C(O)O—R₁; and
(e) —C(O)N(R₄R₅);
alternatively, G and W taken together with the atoms they are attached to a form cyclic structure selected from: 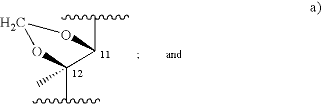 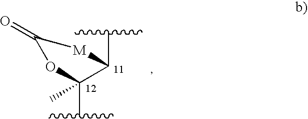
where M is O, —CHR₁ or N-J₁-R₁₂, and where J₁ is absent, O, NH, NHC(O), or N═CH; and R₁₂ is selected from the group consisting of:
i. hydrogen;
ii. R₂; and
iii. R₆;
one of A and B is R₁₃ and the other is OR₁₃, wherein R₁₃ is independently selected from:
(a) hydrogen;
(b) —R₂;
(c) —C(O)R₁;
(d) —C(O)NHR₁;
(e) —S(O)₂R₁;
(f) -monosaccharide; and
(g) -disaccharide;
alternatively, A and B taken together with the carbon atom to which they are attached to form:
(a) C(O);
(b) C═CH-J-R₇, where J is absent, O, C(O), S(O)₂, NH, NHC(O), NHC(O)NH or NHS(O)₂; and wherein R₇ is independently selected from halogen and R₁;
L is independently selected from R₂;
Q is:
(a) —R₁;
(b) —C(O)R₁;
(c) —C(O)NHR₁;
(d) —C(O)OR₁;
(e) —S(O)₂R₁;
(f) monosaccharide;
(g) disaccharide; or
(h) trisaccharide;
Z is:
(a) hydrogen;
(b) —N₃;
(c) —CN;
(d) —NO₂;
(e) —C(O)NH₂;
(f) —C(O)OH;
(g) —CHO;
(h) —R₂;
(i) —C(O)OR₂;
(j)—C(O)R₂; or
(k) —C(O)NR₃R₄;
each of X and Y is independently:
a) hydrogen;
b) hydroxy;
c) halogen; or
d) —R₂; and [symbol]
=either a carbon-carbon single bond or a carbon-carbon double bond.

2. A compound according to claim 1 represented by formula (II): 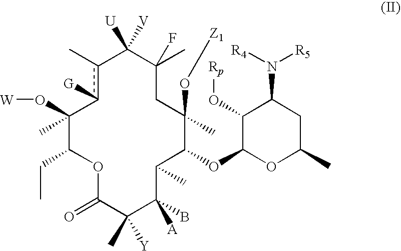
where Rₚ is hydrogen, hydroxy protecting group, ester or hydroxy prodrug; A, B, G, W, U, V, Y, R₄, R₅ and Z₁ are as previously defined in claim 1.

3. A compound according to claim 1 represented by formula (III): 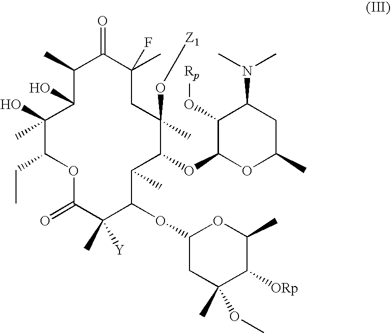
where Rₚ is hydrogen, hydroxy protecting group, ester or hydroxy prodrug; Z₁, Y and Rₚ are as previously defined in claim 1.

4. A compound according to claim 1 represented by formula (IV): 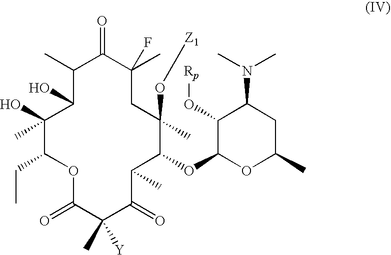
where Rₚ is hydrogen, hydroxy protecting group, ester or hydroxy prodrug; Z₁, Y and Rₚ are as previously defined in claim 1.

5. A compound according to claim 1 represented by formula (V): 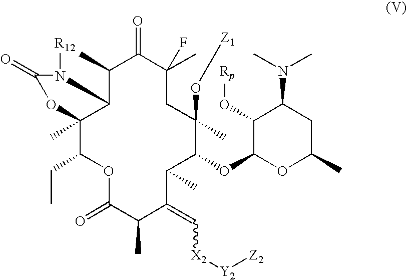
where X₂ is C(O) or CH₂; Y₂ is absent, O, S, NH, C(O), C(O)O, C(O)NH, S(O), S(O)₂, C(S), C(S)NH, OC(O)O, OC(O)NH, OC(O), C(O)O, NHC(O)O, NHC(O), NHC(O)NH, NHS(O)₂, S(O)₂NH, NHS(O)₂NH, or C═N-E-R₃; Z₂ is hydrogen, R₂ or R₆; Rₚ is hydrogen, hydroxy protecting group, ester or hydroxy prodrug; and R₁₂, Z₁ and Y are as previously defined in claim 1.

6. A compound according to claim 1 represented by formula (VI): 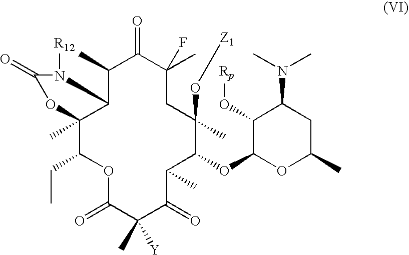
where Rₚ is hydrogen, hydroxy protecting group, ester or hydroxy prodrug; Y, Rₚ, R₁₂ and Z₁ are as previously defined in claim 1.

7. A compound according to claim 1 represented by formula (VII): 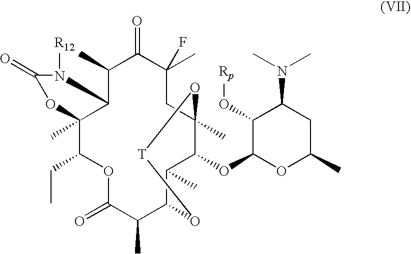
where Rₚ is hydrogen, hydroxy protecting group, ester or hydroxy prodrug; T, R₁₂ and Z₁ are as previously defined in claim 1.

8. A compound of claim 1 having the Formula A, selected from compounds 1-43 of Table 1: 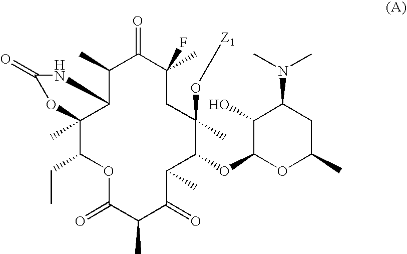
wherein Z1 is delineated for each example in Table 1,
TABLE 1

| Compound | Z₁ |
|---|---|
| 1 | CH₃ |
| 2 | 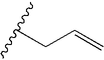 |
| 3 | 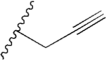 |
| 4 | 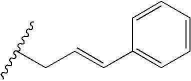 |
| 5 | 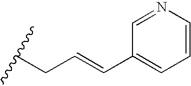 |
| 6 | 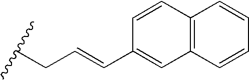 |
| 7 | 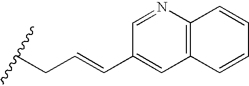 |
| 8 | 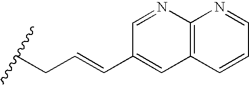 |
| 9 | 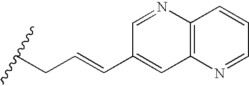 |
| 10 | 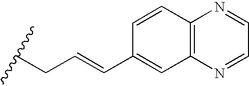 |
| 11 | 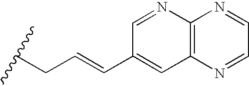 |
| 12 | 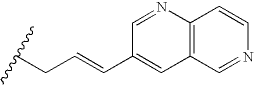 |
| 13 | 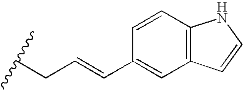 |
| 14 | 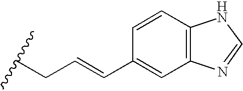 |
| 15 | 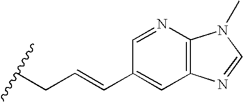 |
| 16 | 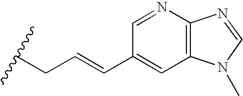 |
| 17 | 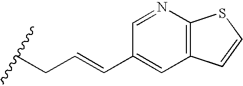 |
| 18 | 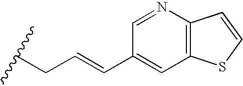 |
| 19 | 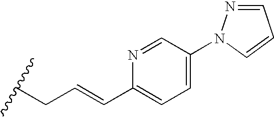 |
| 20 | 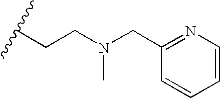 |
| 21 | 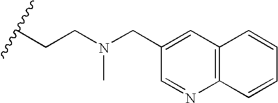 |
| 22 | 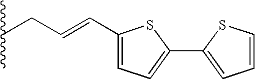 |
| 23 | 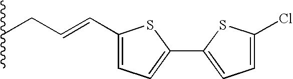 |
| 24 | 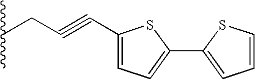 |
| 25 | 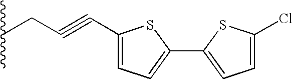 |
| 26 | 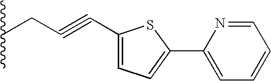 |
| 27 | 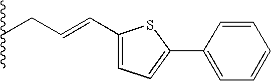 |
| 28 | 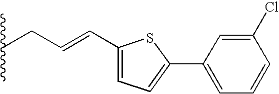 |
| 29 | 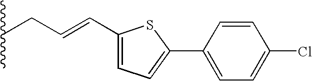 |
| 30 | 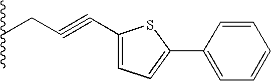 |
| 31 | 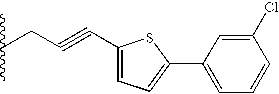 |
| 32 | 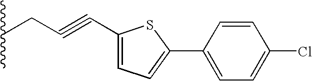 |
| 33 | 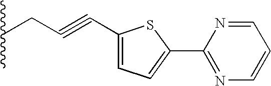 |
| 34 | 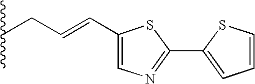 |
| 35 | 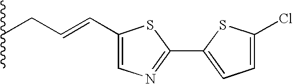 |
| 36 | 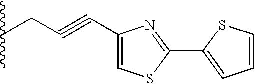 |
| 37 | 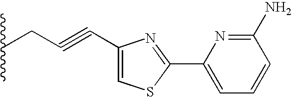 |
| 38 | 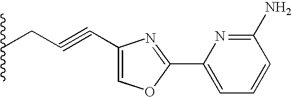 |
| 39 | 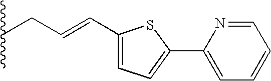 |
| 40 | 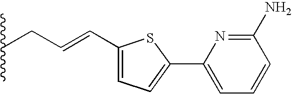 |
| 41 | 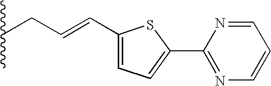 |
| 42 | 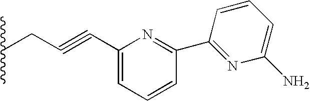 |
| 43 | 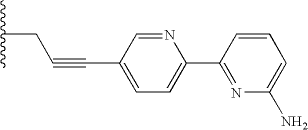 |

9. A compound of claim 1 having the Formula B, selected from compounds 44-55 of Table 2: 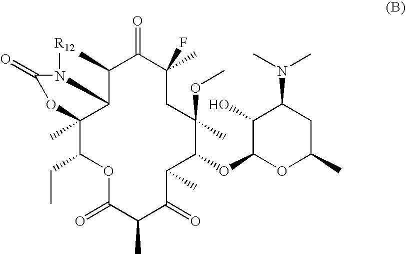
wherein R₁₂ and Z₁ is delineated for each example in Table 2,
TABLE 2

| Com- | |
|---|---|
| pound | R₁₂ |
| 44 | H |
| 45 | 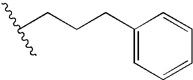 |
| 46 | 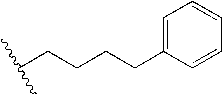 |
| 47 | 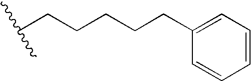 |
| 48 | 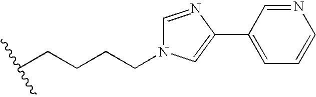 |
| 49 | 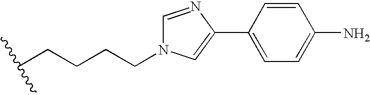 |
| 50 | 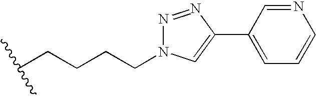 |
| 51 | 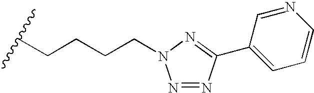 |
| 52 | 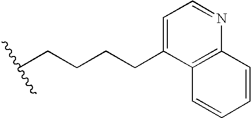 |
| 53 | 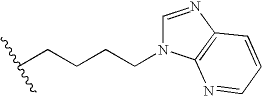 |
| 54 | 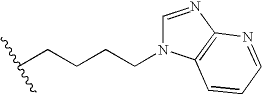 |
| 55 | 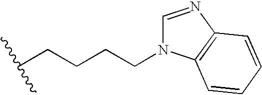 |

10. A pharmaceutical composition comprising a therapeutically effective amount of a compound of claim 1 or a pharmaceutically acceptable salt, ester or prodrug thereof, in combination with a pharmaceutically acceptable carrier.

11. A method for treating a bacterial infection in a subject, comprising administering to said subject a therapeutically effective amount of a pharmaceutical composition according to claim 10.

12. A method of treating cystic fibrosis in subject, comprising administering to said subject, a therapeutically effective amount of a pharmaceutical composition of claim 10.

13. A method of treating inflammation in a subject comprising administering to said subject, therapeutically effective amount of a pharmaceutical composition of claim 10.
